(19) United States
(12) Patent Application Publication
(10) Pub. No.: US 2009/0186815 A1
(43) Pub. Date: Jul. 23, 2009

(54) IDENTIFICATION OF JAK/STAT PATHWAY MODULATING GENES BY GENOME WIDE RNAI SCREENING

(76) Inventors: Michael Boutros, Heidelberg (DE); Martin Zeidler, Göttingen (DE); Patrick Mueller, Göttingen (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 11/917,653

(22) PCT Filed: Jun. 14, 2006
(86) PCT No.: PCT/EP2006/005744

§ 371 Date: Dec. 16, 2008

(30) Foreign Application Priority Data
Jun. 15, 2005 (EP) 05 012 934.5

(51) Int. Cl. A61K 38/14; G01N 33/566; G01N 33/50; C12Q 1/68; G01N 33/536; C12Q 1/02; C07H 21/04; C07K 14/47; C12N 5/06; C12N 15/87; A61K 31/711

(52) U.S. Cl. 514/12; 436/86; 436/94; 435/6; 436/536; 435/29; 536/23.5; 536/24.5; 530/350; 435/375; 435/455; 514/44

(57) ABSTRACT

The present invention relates to a method for identifying a compound capable of modulating the activity of the JAK/STAT pathway and to the use of different JAK/STAT pathway components as a target for the modulation of the activity of the JAK/STAT pathway. Moreover, the present invention is concerned with a method for modulating the activity of the JAK/STAT pathway. Furthermore, the present invention pertains to a pharmaceutical composition and to the use of different JAK/STAT pathway components and/or effector molecules thereof for the manufacture of such composition for the diagnosis, prevention or treatment of a JAK/STAT pathway associated disorder.

The present invention relates to a method for identifying a compound capable of modulating the activity of the JAK/STAT pathway and to the use of different JAK/STAT pathway components as a target for the modulation of the activity of the JAK/STAT pathway. Moreover, the present invention is concerned with a method for modulating the activity of the JAK/STAT pathway. Furthermore, the present invention pertains to a pharmaceutical composition and to the use of different JAK/STAT pathway components and/or effector molecules thereof for the manufacture of such composition for the diagnosis, prevention or treatment of a JAK/STAT pathway associated disorder.

Signalling pathways mediating the transduction of information between cells are essential for development, cellular differentiation and homeostasis (Brivanlou, A. H. & Darnell, J. E., Jr., Science 295, 813-8. (2002)). Their dysregulation is also frequently associated with human malignancies. The JAK/STAT pathway represents one such signalling cascade whose evolutionarily conserved roles include cell proliferation and haematopoiesis (Hombria, J. C. & Brown, S., Curr Biol 12, R569-75 (2002)).

Developmental genetic screens in Drosophila have identified multiple JAK/STAT pathway components on the basis of their segmentation phenotype (Binari, R. & Perrimon, N., Genes Dev 8, 300-12. (1994); Harrison, D. A., McCoon, P. E., Binari, R., Gilman, M. & Perrimon, N., Genes Dev 12, 3252-63. (1998); Hou, X. S., Melnick, M. B. & Perrimon, N., Cell 84, 411-9 (1996)) and subsequent analysis of the pathway has characterised evolutionarily conserved roles during immune responses, haematopoiesis and cellular proliferation (Lagueux, M., Perrodou, E., Levashina, E. A., Capovilla, M. & Hoffmann, J. A., Proc Natl Acad Sci USA 97, 11427-32. (2000); Boutros, M., Agaisse, H. & Perrimon, N., Dev Cell 3, 711-22. (2002); Meister, M. & Lagueux, M., Cell Microbiol 5, 573-580 (2003); Mukherjee, T., Castelli-Gair Hombria, J. & Zeidler, M. P., Oncogene in press (2005)). The JAK/STAT signalling cascade in Drosophila is comprised of the extracellular ligand Unpaired (Upd) (Harrison, D. A., McCoon, P. E., Binari, R., Gilman, M. & Perrimon, N., Genes Dev 12, 3252-63. (1998)), a trans-membrane receptor with homology to the IL6 receptor family termed Domeless (Dome) (Brown, S., Hu, N. & Castelli-Gair Hombria, J., Curr Biol 11, 1700-5. (2001)), a single Janus tyrosine kinase (JAK) called Hopscotch (Hop) (Binari, R. & Perrimon, N., Genes Dev 8, 300-12. (1994)) and the STAT92E transcription factor (Hou, X. S., Melnick, M. B. & Perrimon, N., Cell 84, 411-9 (1996); Yan, R., Small, S., Desplan, C., Dearolf, C. R. & Darnell, J. E., Jr., Cell 84, 421-30 (1996)) (FIG. 1a). Known regulators of JAK/STAT signalling including a family of SOCS-like genes (Callus, B. A. & Mathey-Prevot, B.; Oncogene 21, 4812-4821 (2002); Karsten, P., Hader, S. & Zeidler, M. P., Mech Dev 117, 343-6 (2002)), dPIAS/Su(var)2-10 (Betz, A., Lampen, N., Martinek, S., Young, M. W. & Darnell, J. E., Jr., Proc Natl Acad Sci USA 98, 9563-8 (2001)) and STAM (Mesilaty-Gross, S., Reich, A., Motro, B. & Wides, R., Gene 231, 173-86 (1999)) are functionally conserved and were identified based on their homology to components originally characterised in mammalian cell culture studies (Hombria, J. C. & Brown, S., Curr Biol 12, R569-75 (2002)). Although successful in identifying the pathway members Upd, Dome, Hop and STAT92E, it is probable that forward genetic approaches have missed components possibly due to non-saturating mutagenesis, genetic redundancy or phenotypic pleiotropy (Nagy, A., Perrimon, N., Sandmeyer, S. & Plasterk, R., Nat Genet 33 Suppl, 276-84 (2003)).

In order to identify novel pathway components and circumvent limitations of classical genetic screens, the inventors of the present invention have undertaken a genome-wide RNA interference (RNAi) screen, a powerful technique for the identification of new components of diverse cellular pathways (Kamath, R. S. et al., Nature 421, 231-7 (2003); Kittler, R. et al., Nature 432, 1036-40 (2004); Berns, K. et al., Nature 428, 431-7 (2004); Paddison, P. J. et al., Nature 428, 427-31 (2004); Boutros, M. et al., Science 303, 832-5 (2004)). Using this screen, a systematic genome-wide survey for genes required for JAK/STAT pathway activity could be performed. Analysis of 20,026 RNAi-induced phenotypes in cultured Drosophila melanogaster haemocyte-like cells identified interacting genes encoding 4 known and 84 previously uncharacterised proteins. Subsequently, cell based epistasis experiments have been used to classify these based on their interaction with known components of the signalling cascade. In addition to multiple human disease gene homologues, the inventors of the present invention have identified the tyrosine phosphatase Ptp61F and the Drosophila homologue of BRWD3, a bromo-domain containing protein disrupted in leukaemia. Moreover, in vivo analysis demonstrates that disrupted dBRWD3 and overexpressed Ptp61F function as suppressors of leukaemia-like blood cell tumours. This screen represents a comprehensive identification of novel loci required for JAK/STAT signalling and provides molecular insights into an important pathway relevant for human diseases.

A first aspect of the present invention, therefore, relates to a method for identifying a compound capable of modulating the activity of the JAK/STAT pathway, comprising

(a) contacting a compound with at least one target molecule selected from
(i) nucleic acid molecules, comprising
   (i.1) a nucleotide sequence as shown in SEQ ID NOs. 88 to 265;
   (i.2) a nucleotide sequence which is complementary to a nucleotide sequence of (i.1);
   (i.3) a nucleotide sequence which has an identity of at least 65% to a nucleotide sequence of (i.1) or (i.2); and/or
   (i.4) a nucleotide sequence which hybridizes under stringent conditions to a nucleotide sequence of (i.1), (i.2) or (i.3); and
(ii) polypeptide molecules
   (ii.1) encoded by the nucleic acid molecules of (i) and/or
   (ii.2) having the sequences as shown in SEQ ID NOs. 1-87, and
      (b) determining the degree of modulation of the at least one target molecule by the compound.

In accordance with the present invention, it is to be understood, that the term “modulating the activity of the JAK/STAT pathway”, when used herein, means activating or inhibiting the activity of the JAK/STAT signalling pathway. An activation or inhibition of the activity of the JAK/STAT signalling pathway may e.g. be mediated by an activation or inhibition of at least one component of the JAK/STAT pathway, either directly or indirectly.

According to the present invention, step (a) of the method for identifying a compound capable of modulating the activity of the JAK/STAT pathway comprises contacting a compound with at least one target molecule selected from the nucleic acid molecules of (i) and the polypeptide molecules of (ii).

The nucleic acid molecules of (i) used according to the method for identifying a compound capable of modulating the activity of the JAK/STAT pathway comprise in one embodiment of the present invention a nucleotide sequence of (i.1) as show in SEQ ID NOs. 88 to 265. Preferably, the nucleic acid molecules of (i) comprise a nucleic acid sequence of (i.1) as shown in SEQ ID NOs. 88 to 174. More preferably, the nucleic acid molecules of (i) comprise a nucleic acid sequence of (i.1) as shown in SEQ ID NOs. 91, 116, 124, 133, 136, 152, 154, 155 to 174.

It is to be understood that the Drosophila gene sequences of SEQ ID Nos. 175-265 encompasse respective splice variants.

Moreover, nucleic acid molecules of (i) used according to the method for identifying a compound capable of modulating the activity of the JAK/STAT pathway comprise in another embodiment of the present invention a nucleotide sequence of (i.2) which is complementary to a nucleotide sequence of (i.1). Preferably, the nucleic acid molecules of (i) comprise a nucleic acid sequence of (i.2) which is complementary to a nucleotide sequence of (i.1) as shown in SEQ ID NOs. 88 to 174. More preferably, the nucleic acid molecules of (i) comprise a nucleic acid sequence of (i.2) which is complementary to a nucleotide sequence of (i.1) as shown in SEQ ID NOs. 91, 116, 124, 133, 136, 152, 154, 155 to 174.

In a further embodiment of the present invention, the nucleic acid molecules of (i) used according to the method for identifying a compound capable of modulating the activity of the JAK/STAT pathway comprise a nucleotide sequence of (i.3) which has an identity of at least 65, preferably at least 70, more preferably at least 75 and most preferably at least 80% to a nucleotide sequence of (i.1) or (i.2). Within the context of the present application, the term “has an identity of at least 65, preferably at least 70, more preferably at least 75 and most preferably at least 80%”, as used herein, means that the sequence identity is at least 65, 66, 67, 6, 69, preferably at least 70, 71, 72, 73, 74, more preferably at least 75, 76, 77, 78, 79 and most preferably at least 80, 81, 82, 83, 84, 85, 86, 87, 88, 89, 90, 91, 92, 93, 94, 95, 96, 97, 98, 99 or 100%. Preferably, the nucleic acid molecules of (i) comprise a nucleotide sequence of (i.3) which has an identity of at least 65, preferably at least 70, more preferably at least 75 and most preferably at least 80% to a nucleotide sequence of (i.1) as shown in SEQ ID NOs. 88 to 174 or a nucleotide sequence of (i.2) which is complementary to a nucleotide sequence of (i.1) as shown in SEQ ID NOs. 88 to 174. More preferable, the nucleic acid molecules of (i) comprise a nucleotide sequence of (i.3) which has an identity of at least 65, preferably at least 70, more preferably at least 75 and most preferably at least 80% to a nucleotide sequence of (i.1) as shown in SEQ ID NOs. 91, 116, 124, 133, 136, 152, 154, 155 to 174 or a nucleotide sequence of (i.2) which is complementary to a nucleotide sequence of (i.1) as shown in SEQ ID NOs. 91, 116, 124, 133, 136, 152, 154, 155 to 174.

Finally, the nucleic acid molecules of (i) used according to the method for identifying a compound capable of modulating the activity of the JAK/STAT pathway comprise in a further embodiment of the present invention a nucleotide sequence of (i.4) which hybridizes under stringent conditions to a nucleotide sequence of (i.1), (i.2) or (i.3). The term “hybridizes under stringent conditions” according to the present application is used as described in Sambrook et al. Molecular Cloning, A Laboratory Manual, Cold Spring Harbor, Laboratory Press (1989), 1.101-1.104. Consequently, hybridization under stringent conditions occurs when a positive hybridization signal is still detected after washing for 1 h with 1×SSC and 0.1% SDS at 55° C., preferably at 62° C. and most preferably at 68° C., in particular for 1 h in 0.2×SSC and 0.1% SDS at 55° C., preferably at 62° C. and most preferably at 68° C. It is preferred that the nucleic acid molecules of (i) comprise a nucleotide sequence of (i.4) which hybridizes under stringent conditions to a nucleotide sequence of (i.1) as shown in SEQ ID NOs. 88 to 174, a nucleotide sequence of (i.2) which is complementary to a nucleotide sequence of (i.1) as shown in SEQ ID NOs. 88 to 174 or a nucleotide sequence of (i.3) which has an identity of at least 65, preferably at least 70, more preferably at least 75 and most preferably at least 80% to a nucleotide sequence of (i.1) as shown in SEQ ID NOs. 88 to 174 or a nucleotide sequence of (i.2) which is complementary to a nucleotide sequence of (i.1) as shown in SEQ ID NOs. 88 to 174. More preferably, the nucleic acid molecules of (i) comprise a nucleotide sequence of (i.4) which hybridizes under stringent conditions to a nucleotide sequence of (i.1) as shown in SEQ ID NOs. 91, 116, 124, 133, 136, 152, 154, 155 to 174, a nucleotide sequence of (i.2) which is complementary to a nucleotide sequence of (i.1) as shown in SEQ ID NOs. 91, 116, 124, 133, 136, 152, 154, 155 to 174 or a nucleotide sequence of (i.3) which has an identity of at least 65, preferably at least 70, more preferably at least 75 and most preferably at least 80% to a nucleotide sequence of (i.1) as shown in SEQ ID NOs. 91, 116, 124, 133, 136, 152, 154, 155 to 174 or a nucleotide sequence of (i.2) which is complementary to a nucleotide sequence of (i.1) as shown in SEQ ID NOs. 91, 116, 124, 133, 136, 152, 154, 155 to 174.

The nucleic acid molecules of (i) used according to the method for identifying a compound capable of modulating the activity of the JAK/STAT pathway may be present in single-stranded or double-stranded form and may be selected from RNA, DNA or nucleic acid analog molecules, such as sugar- and backbone-modified ribonucleic acids or deoxyribonucleic acids. It should be noted, however, that other nucleic acid analogs, such as peptide nucleic acids (PNA) or locked nucleic acids (LNA), are also suitable.

Moreover, according to the present invention, the nucleic acid molecules of (i) used according to the present invention may be non-recombinant nucleic acid molecules, recombinant nucleic acid molecules generated by recombinant methods, e.g. by known amplification procedures such as PCR, or chemically synthesized nucleic acid molecules. The nucleic acid molecules of (i) may be present in isolated, i.e. purified, form or in non-isolated form, i.e. in a cellular environment.

In a preferred embodiment of the present invention, the nucleic acid molecules of (i) used according to the present invention are present in a vector, which may be any prokaryotic or eukaryotic vector, on which the nucleic acid sequence is present preferably under control of a suitable expression signal, e.g. promoter, operator, enhancer etc. Examples for prokaryotic vectors are chromosomal vectors, such as bacteriophages, and extrachromosomal vectors, such as plasmids, wherein circular plasmid vectors are preferred. Examples for eukaryotic vectors are yeast vectors or vectors suitable for higher cells, e.g. insect cells or mammalian cells, plasmids or viruses.

The polypeptide molecules of (ii) used according to the method for identifying a compound capable of modulating the activity of the JAK/STAT pathway are encoded by the nucleic acid molecules of (i) described above and or have a sequence as shown in SED ID Nos. 1-87. According to a preferred embodiment of the present invention, the polypeptide molecules of (ii) have an amino acid sequence as shown in SEQ ID NO. 4, 29, 37, 46, 49, 65, 67 to 87.

The compound used in step (a) of the method for identifying a compound capable of modulating the activity of the JAK/STAT pathway may be selected from compounds capable of directly and/or indirectly inhibiting or activating the transcription or translation of a nucleic acid molecule of (i). Preferably, the compounds capable of directly and/or indirectly inhibiting or activating the transcription or translation of a nucleic acid molecule of (i) comprise polypeptides such as proteins, enzymes, antibodies, polypeptide inhibitors, polypeptide activators, agonist, antagonists, mimetics, low molecular weight substances, antisense molecules, RNAi molecules and ribozymes. More preferably, the compounds capable of directly and/or indirectly inhibiting or activating the transcription or translation of a nucleic acid molecule of (i) are antisense molecules directed against a nucleic acid molecule of (i) or RNAi molecules. The antisense molecules and RNAi molecules may be prepared by any method known in the art for the synthesis of nucleic acid molecules. These include techniques for chemically synthesising oligonucleotides such as solid phase phosphoramidite chemical synthesis. Alternatively, said molecules may be generated by in vitro and in vivo transcription of DNA sequences.

Moreover, the compound used in step (a) of the method for identifying a compound capable of modulating the activity of the JAK/STAT pathway may also be selected from compounds capable of directly and/or indirectly inhibiting or activating a polypeptide molecule of (ii). Preferably, the compounds capable of directly and/or indirectly inhibiting or activating a polypeptide molecule of (ii) comprise polypeptides such as proteins, enzymes, antibodies, polypeptide inhibitors, polypeptide activators, agonist, antagonists, mimetics, oligopeptides, low molecular weight substances and polypeptide cofactors. More preferably, the compounds capable of directly and/or indirectly inhibiting or activating a polypeptide molecule of (ii) are antibodies or fragments thereof directed against a polypeptide molecule of (ii). Within the context of the present invention, the term “antibody”, as used herein, encompasses polyclonal antibodies, monoclonal antibodies, e.g. chimeric antibodies, humanized antibodies, human antibodies or recombinant antibodies, e.g. single-chain antibodies. Further, the term “antibody fragment” encompasses common antibody fragments, e.g. proteolytic fragments such as Fab, F(ab)₂, Fab′ or recombinant fragments such as scFv. The antibodies or fragments thereof may be obtained using hybridoma cell lines or recombinant DNA methods using techniques well known in the art. However, the antibodies or fragments thereof may also be isolated from phage antibody libraries using techniques described in the art.

According to the present invention, step (b) of the method for identifying a compound capable of modulating the activity of the JAK/STAT pathway comprises determining the degree of modulation of the at least one target molecule by the compound. Preferably, the degree of modulation of the at least one target molecule by the compound may be determined either by measuring the amount and/or expression rate of the nucleic acid molecules of (i) or by measuring the amount and/or activity of the polypeptide molecules of (ii). A variety of protocols including, for example, ELISA, RIA, and FACS, for measuring nucleic acid molecules and/or proteins are known in the art and provide a basis for measuring the amount and/or expression rate of a nucleic acid molecule or the amount and/or activity of a polypeptide molecule. Particularly, the capability of a substance to modulate the activity of the JAK/STAT pathway is determined as described in the Example.

According to the present invention, the method for identifying a compound capable of modulating the activity of the JAK/STAT pathway may be a molecular based assay or a cellular assay. Therefore, the at least one target molecule may be provided either in vivo in a cellular system, preferably a cellular system overexpressing the at least one target molecule, or in vitro in cell fractions containing the at least one target molecule or with the at least one target molecule in a substantially isolated and purified form. Methods for providing the at least one target molecule are well known in the art and may be used in performing the present invention. According to the present invention, it is preferred that the method for identifying a compound capable of modulating the activity of the JAK/STAT pathway is performed in a high-throughput format.

A second aspect of the present invention pertains to the use of at least one molecule selected from

(i) nucleic acid molecules, comprising
(i.1) a nucleotide sequence as shown in SEQ ID NOs. 88 to 265;
(i.2) a nucleotide sequence which is complementary to a nucleotide sequence of (i.1);
(i.3) a nucleotide sequence which has an identity of at least 65% to a nucleotide sequence of (i.1) or (i.2); and/or
(i.4) a nucleotide sequence which hybridizes under stringent conditions to a nucleotide sequence of (i.1), (i.2) or (i.3); and
   (ii) polypeptide molecules
(ii.1) encoded by the nucleic acid molecules of (i) and/or
(ii.2) having the sequences as shown in SEQ ID NOs. 1-87,
   as a target for the modulation of the activity of the JAK/STAT pathway.

Within the context of the present invention, the nucleic acid molecules of (i), comprising (i.1) a nucleotide sequence as shown in SEQ ID NOs. 88 to 265, (i.2) a nucleotide sequence which is complementary to a nucleotide sequence of (i.1), (i.3) a nucleotide sequence which has an identity of at least 65% to a nucleotide sequence of (i.1) or (i.2), and/or (i.4) a nucleotide sequence which hybridizes under stringent conditions to a nucleotide sequence of (i.1), (i.2) or (i.3), and the polypeptide molecules of (ii) encoded by the nucleic acid molecules of (i) used as afore-mentioned are as described above.

A third aspect of the present invention relates to a method for modulating the activity of the JAK/STAT pathway comprising contacting a cell with at least one molecule selected from

(i) nucleic acid molecules, comprising
(i.1) a nucleotide sequence as shown in SEQ ID NOs. 88 to 265;
(i.2) a nucleotide sequence which is complementary to a nucleotide sequence of (i.1);
(i.3) a nucleotide sequence which has an identity of at least 65% to a nucleotide sequence of (i.1) or (i.2); and/or
(i.4) a nucleotide sequence which hybridizes under stringent conditions to a nucleotide sequence of (i.1), (i.2) or (i.3);
   (ii) polypeptide molecules
(ii.1) encoded by the nucleic acid molecules of (i) and/or
(ii.2) having the sequences as shown in SEQ ID NOs. 1-87; and
   (iii) effector molecules of (i) and/or (ii).

The method for modulating the activity of the JAK/STAT pathway may suitably be performed as molecular based assay or cellular assay. Preferably, the cell used in the method for modulating the activity of the JAK/STAT pathway is a cell showing the JAK/STAT pathway, e.g. an animal cell.

According to the present invention, the nucleic acid molecules of (i), comprising (i.1) a nucleotide sequence as shown in SEQ ID NOs. 88 to 265, (i.2) a nucleotide sequence which is complementary to a nucleotide sequence of (i.1), (i.3) a nucleotide sequence which has an identity of at least 65% to a nucleotide sequence of (i.1) or (i.2), and/or (i.4) a nucleotide sequence which hybridizes under stringent conditions to a nucleotide sequence of (i.1), (i.2) or (i.3), and the polypeptide molecules of (ii) encoded by the nucleic acid molecules of (i) used according to the method for modulating the activity of the JAK/STAT pathway are as described above.

Moreover, the effector molecules of (i) and/or (ii) used according to the method for modulating the activity of the JAK/STAT pathway are selected from polypeptides such as proteins, enzymes, antibodies, polypeptide inhibitors, polypeptide activators, agonist, antagonists, mimetics, oligopeptides, cofactors, low molecular weight substances, antisense molecules, RNAi molecules and ribozymes. Preferably, the effector molecules of (i) and/or (ii) are compounds identified by the method for identifying compounds of modulating the activity of the JAK/STAT pathway described above. More preferably, the effector molecules of (i) and/or (ii) are antibodies or fragments thereof directed against a polypeptide molecule of (ii), antisense molecules directed against a nucleic acid molecule of (i) and/or RNAi molecules.

Further, the present invention is concerned in a fourth aspect with a pharmaceutical composition comprising as an active agent at least one molecule selected from

(i) nucleic acid molecules, comprising
(i.1) a nucleotide sequence as shown in SEQ ID NOs. 88 to 265;
(i.2) a nucleotide sequence which is complementary to a nucleotide sequence of (i.1);
(i.3) a nucleotide sequence which has an identity of at least 65% to a nucleotide sequence of (i.1) or (i.2); and/or
(i.4) a nucleotide sequence which hybridizes under stringent conditions to a nucleotide sequence of (i.1), (i.2) or (i.3);
   (ii) polypeptide molecules
(ii.1) encoded by the nucleic acid molecules of (i) and/or
(ii.2) having the sequences as shown in SEQ ID NOs. 1-87; and
   (iii) effector molecules of (i) and/or (ii).

According to the present invention, the nucleic acid molecules of (i), comprising (i.1) a nucleotide sequence as shown in SEQ ID NOs. 88 to 265, (i.2) a nucleotide sequence which is complementary to a nucleotide sequence of (i.1), (i.3) a nucleotide sequence which has an identity of at least 65% to a nucleotide sequence of (i.1) or (i.2), and/or (i.4) a nucleotide sequence which hybridizes under stringent conditions to a nucleotide sequence of (i.1), (i.2) or (i.3), the polypeptide molecules of (ii) encoded by the nucleic acid molecules of (i) and the effector molecules (of (iii)) of (i) and/or (ii) comprised in the pharmaceutical composition of the invention are as described above.

In addition to the at least one active ingredient, the pharmaceutical composition of the invention may contain suitable pharmaceutically acceptable carriers, diluents and/or adjuvants, which facilitate processing of the active ingredient into preparations, which can be used pharmaceutically. Further details on techniques for formulation and administration may be found in the latest edition of Remington's Pharmaceutical Sciences (Maack Publishing Co., Easton, Pa.).

The pharmaceutical composition of the present invention is particularly suitable for the diagnosis, prevention or treatment of a JAK/STAT pathway associated disorder. Preferably, the JAK/STAT pathway associated disorder is selected from the group consisting of papillary thyroid carcinoma, Refsum disease, blood-brain barrier glucose transport defect, X-linked nonsyndromic mental retardation, long QT syndrome 4, subcortical laminar heterotopia, leukemia, steroid-resistant nephrotic syndrome, invasive pituitary tumor, sporadic Sotos syndrome, autosomal dominant iron overload, hereditary pancreatitis, stomatocytosis I, atypical Rett syndrome, phosphoglycerate dehydrogenase deficiency, Wolman disease, neurophysiologic defect in schizophrenia, autosomal recessive SCID (T-negative/B-positive type), atelostogenesis (type I), Larson syndrome, spondylocarpotarsal synostosis syndrome, frontometaphyseal dysplasia, diabetes mellitus (type II), susceptibility to insulin resistance, Griscelli Syndrome, limb-girdle muscular dystrophy (type 2A), growth hormone insensitivity with immunodeficiency and breast cancer.

In one embodiment of the present invention the pharmaceutical composition is used for the prevention or treatment of a JAK/STAT pathway associated disorder. Pharmaceutical compositions suitable for the prevention or treatment of a JAK/STAT pathway associated disorder include compositions wherein the at least one active ingredient is contained in an effective amount to achieve the intended purpose. The determination of an effective dose is well within the capability of those skilled in the art. For any compounds, the therapeutically effective dose can be estimated initially either in cell culture assays or in animal models, usually mice, rabbits, dogs or pigs. The animal model may also be used to determine the appropriate concentration range and route of administration. Such information can then be used to determine useful doses and routes for administration in humans.

The actual amount of the pharmaceutical composition administered, will of course, be dependent on the subject being treated, on the subject's weight, the severity of the JAK/STAT pathway associated disorder, the manner of administration and the judgement of the prescribing physician. For the pharmaceutical composition of the invention, a daily dosage of 1 to 200 mg of the at least one active ingredient per kg and day, particularly 10 to 100 mg of the at least one active ingredient per kg and day, is suitable. Suitable routes of administration may, for example, include oral, intravenous, intramuscular, intraarterial, intramedullary, intrathecal, intraventricular, transdermal, subcutaneous, intraperitoneal, intranasal, enteral, topical, sublingual or rectal administrations. Preferably, the subject being treated is an animal, in particular a human being.

In another embodiment of the present invention the pharmaceutical composition is used for the diagnosis of a JAK/STAT pathway associated disorder, e.g. a disorder characterized by or associated with the over- or underexpression of a nucleic acid molecule of (i) or a polypeptide molecule of (ii). Diagnostic assays include methods which utilize the pharmaceutical composition and a label to detect the nucleic acid molecule of (i) or polypeptide molecule of (ii) in human body fluids or extracts of cells or tissues.

Finally, a further aspect of the present invention relates to the use of at least one molecule selected from

(i) nucleic acid molecules, comprising
(i.1) a nucleotide sequence as shown in SEQ ID NOs. 88 to 265;
(i.2) a nucleotide sequence which is complementary to a nucleotide sequence of (i.1);
(i.3) a nucleotide sequence which has an identity of at least 65% to a nucleotide sequence of (i.1) or (i.2); and/or
(i.4) a nucleotide sequence which hybridizes under stringent conditions to a nucleotide sequence of (i.1), (i.2) or (i.3);
   (ii) polypeptide molecules
(ii.1) encoded by the nucleic acid molecules of (i) and/or
(ii.2) having the sequences as shown in SEQ ID NOs. 1-87; and
   (iii) effector molecules of (i) and/or (ii);
   for the manufacture of a pharmaceutical composition for the diagnosis, prevention or treatment of a JAK/STAT pathway associated disorder.

According to the present invention, the nucleic acid molecules of (i), comprising (i.1) a nucleotide sequence as shown in SEQ ID NOs. 88 to 265, (i.2) a nucleotide sequence which is complementary to a nucleotide sequence of (i.1), (i.3) a nucleotide sequence which has an identity of at least 65% to a nucleotide sequence of (i.1) or (i.2), and/or (i.4) a nucleotide sequence which hybridizes under stringent conditions to a nucleotide sequence of (i.1), (i.2) or (i.3), the polypeptide molecules of (ii) encoded by the nucleic acid molecules of (i) and the effector molecules (of (iii)) of (i) and/or (ii) used according to the present invention for the manufacture of a pharmaceutical composition for the diagnosis, prevention or treatment of a JAK/STAT pathway associated disorder are as described above.

Moreover, according to the present invention, the pharmaceutical composition and the JAK/STAT pathway associated disorder are as described above.

Methods for the manufacture of a pharmaceutical composition, comprising the step of admixing at least one molecule selected from nucleic acid molecules of (i), comprising (i.1) a nucleotide sequence as shown in SEQ ID NOs. 88 to 265, (i.2) a nucleotide sequence which is complementary to a nucleotide sequence of (i.1), (i.3) a nucleotide sequence which has an identity of at least 65% to a nucleotide sequence of (i.1) or (i.2), and/or (i.4) a nucleotide sequence which hybridizes under stringent conditions to a nucleotide sequence of (i.1), (i.2) or (i.3), polypeptide molecules of (ii) encoded by the nucleic acid molecules of (i) and effector molecules (of (iii)) of (i) and/or (ii) with a pharmaceutically acceptable excipient, vehicle or carrier and optionally other ingredients are well known to those skilled in the art and may be used in performing the present invention.

Further, the present invention shall be explained by the following Tables, Figures and Example.

### Tables

Table 1 shows the RNAi JAK/STAT phenotypes.

Table 2 shows the functional groups classified by InterPro prediction and GO.

Table 3 shows the genetic interactions with hop^{Tuml}.

Table 4 shows sequence and cytological information.

Table 5 shows human homologues of Drosophila genes with JAK/STAT phenotypes.

Table 6 shows human disease homologues of Drosophila genes with JAK/STAT phenotypes.

Supplementary Table 7 shows the expected and observed phenotype frequency.

Table 7 shows preferred human JAK/STAT homologues ranked according to their involvement in a human disease.

Table 8 shows evolutionary and functional conservation of JAK/STAT pathway components.

### FIGURES

FIG. 1 shows the genome-wide RNAi screen for JAK/STAT signalling factors. a: Schematic representation of the Drosophila JAK/STAT signalling pathway. b: Knock-down of known JAK/STAT components leads to loss of pathway induction by Upd whereas knock-down of lacZ, toll and relish show no effect. The red line indicates a 70-fold reporter induction relative to negative control dsRNA. Error bars represent standard deviations of six experiments. c: Screening approach. 20,026 dsRNA were screened in duplicate in 384-well plates prior to computational analysis and retesting. FL: firefly luciferase (indicated in red); RL: Renilla luciferase (indicated in yellow) d: Q-Q plot of normally distributed quantiles against actual screening result quantiles in the pathway reporter channel. A perfect fit to a normal distribution is represented by the red line. Tails of positively and negatively interacting dsRNAs at each extreme with a z-score threshold of >2 and <−2 represent RNAi experiments with significant phenotypes (p<0.05).

FIG. 2 shows the analysis of JAK/STAT activity modulators. a: Schematic representation of positive (red) and negative (green) regulator loci distributed within the Drosophila genome. An interactive version of this panel is available at http://www.dkfz.de/signaling/jak-pathway/cytomap.php. b: Distribution of predicted gene functions. c: Epistasis analysis of the indicated positive pathway regulators showing interactions graded from none (yellow) to strong (red). Results shown have been obtained in two independent octuplicate experiments. Upd: Upd ectopic expression; Upd-CM: Upd conditioned medium; hop^{Tuml}: expression of a constitutively active JAK-allele. Colour coding of z-scores is shown in the key.

FIG. 3 shows that dBRWD3 functions as a JAK/STAT pathway component. a: Domain structure and sequence similarity of Drosophila and human BRWD3 proteins. Percentages show the similarity in the amino acid sequence and regions targeted by two independent dsRNAs independently recovered in the screen are shown. b: Adult Drosophila heads heterozygous for the GMR-updA3′ transgene crossed to wild type (left), stat92E (middle) and dBRWD3 mutants (right). Note the strong reduction in eye size following removal of pathway components. c: hop^{Tuml} induced tumour formation is significantly decreased in both size and frequency of tumours in stat92E and dBRWD3 heterozygous backgrounds. d: By comparison to adult wild type wings (left), ectopic wing vein material (arrow) is present in homozygous dBRWD3^{Δ10} mutant (a putative hypomorphic allele, right), a phenotype reminiscent of the stat92EH^{HJ} mutant.

FIG. 4 shows that Ptp61F is a tumour suppressor in vivo. a: Epistasis analysis of ptp61F dsRNA in cell culture revealed that it acts downstream of Hop and upstream or parallel to STAT92E. b: Haemocyte specific misexpression of ptp61F can protect hop^{Tuml} mutants from melanotic tumour formation. Compare large black tumours in controls (arrow heads, left) with small tumours present in ptp61F expressing individual (right). c: Quantitative analysis of large tumour formation in hop^{Tuml} mutants expressing cytoplasmic Ptp61Fa and nuclear Ptp61Fc shows specificity of rescue for the nuclear isoform (left), an effect that is mirrored by over-expression of the same isoforms in tissue culture based reporter assays (right). Error bars represent standard deviations of 3 or 4 independently tested transgenic lines or eight parallel cell culture experiments.

FIG. 5 shows an overview of primary RNAi screen data. a: False colour representation of the genome-wide screen showing averaged z-scores for each well present in the fifty seven 384-well duplicate plates. Key indicates the colours associated with the z-scores: −4 (red) represents a strong decrease in reporter activity, +4 (blue) represents an increase in activity. Four controls were included in the top left corner of each plate and are visible in all plates except 1 and 9 for which these dsRNA controls failed. b: False colour representation of average z-scores for a representative example from the genome-wide screen (plate 34). Controls present in the lop left corner of each plate were hop (A1), dome (A2), stat92E (B1) and socs36E (B2). dsRNAs from the library were present in all other wells including position B07 which targets hopscotch. L10 which targets CG2033 was excluded from the final list because of a cell viability phenotype previously identified in both Kc and S2R+ cells (Boutros, M. et al., Science 303, 832-5 (2004)). Similarly, 102 and G20 (which both target sbr/CG17335) were excluded due to variability in retesting and a previously described bi-nucleate phenotype (Kiger, A. A. et al., J Biol 2, 27 (2003)). Colour coding for z-scores is shown in the key and uses the same scheme shown in a. c: Histogram of z-scores for the genome-wide screen indicates that the majority of dsRNA experiments do not modify JAK/STAT signaling activity.

FIG. 6 shows the loss of JAK/STAT pathway components and hop^{Tuml} induced tumour formation. hop^{Tuml}/+; +/+ females (top) frequently contain large black melanotic tumours (arrows). hop^{Tuml}/+;stat92E⁰⁶³⁴²/+ heterozygotes which lack one copy of stat92E (middle) contain fewer and smaller tumours. hop^{Tuml}/+;dBRWD3⁰⁵⁸⁴²/+ (bottom) also contain fewer and smaller tumours. Flies were grown in parallel independent experiments at 25° C. and are representative examples of the individuals recovered (see Table 3 for further information).

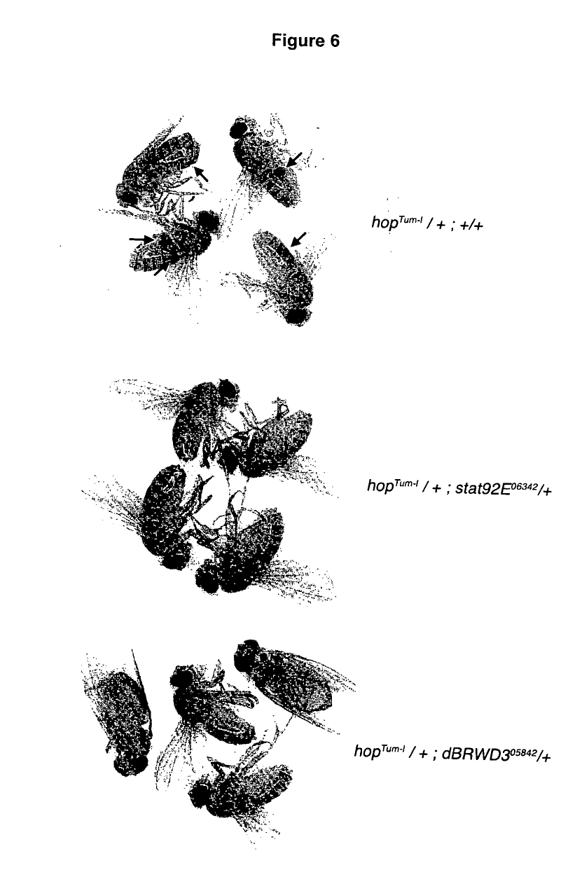

FIG. 7 shows a heat-map showing human JAK/STAT pathway regulating genes identified. Data shown are: the original Drosophila interactions (col 1) expressed as z-scores, fold change in the expression levels of STAT1 and STAT3 target genes (col 2 & 3, respectively) and the levels of phosphorylated STAT1 and STAT3 (col 4 & 5, respectively). In all columns black represents a decrease, white an increase and grey no change in activity.

### EXAMPLE

Signalling pathways mediating the transduction of information between cells are essential for development, cellular differentiation and homeostasis (Brivanlou, A. H. & Darnell, J. E., Jr., Science 295, 813-8. (2002)). Their dysregulation is also frequently associated with human malignancies. The JAK/STAT pathway represents one such signalling cascade whose evolutionarily conserved roles include cell proliferation and haematopoiesis (Hombria, J. C. & Brown, S., Curr Biol 12, R569-75 (2002)). Here, the inventors of the present invention describe a systematic genome-wide survey for genes required for JAK/STAT pathway activity. Analysis of 20,026 RNAi-induced phenotypes in cultured Drosophila melanogaster haemocyte-like cells identified interacting genes encoding 4 known and 84 previously uncharacterised proteins. Subsequently, cell based epistasis experiments have been used to classify these based on their interaction with known components of the signalling cascade. In addition to multiple human disease gene homologues, the inventors of the present invention have identified the tyrosine phosphatase Ptp61F and the Drosophila homologue of BRWD3, a bromo-domain containing protein disrupted in leukaemia (Kalla, C. et al., Genes Chromosomes Cancer 42, 128-43 (2005)). Moreover, in vivo analysis demonstrates that disrupted dBRWD3 and overexpressed Ptp61F function as suppressors of leukaemia-like blood cell tumours. This screen represents a comprehensive identification of novel loci required for JAK/STAT signalling and provides molecular insights into an important pathway relevant for human diseases.

### 1. Experimental Procedures

### 1.1. Constructs and Pathway Reporter

The JAK/STAT firefly luciferase reporter 6x2xDrafLuc was constructed by multimerisation of a molecularly characterised STAT92E binding site present in the promoter of the endogenous target genes Draf (Kwon, E. J. et al., J Biol Chem 275, 19824-19830 (2000)) while the 4xsocsLuc reporter is based on a single region containing four potential STAT92E binding sites present within the first intron of socs36E (Karsten, P., Hader, S. & Zeidler, M. P., Mech Dev 117, 343-6 (2002)). A Renilla luciferase reporter gene under the control of the constitutively active Actin5C promoter was co-transfected and used to monitor cell number.

Strictly speaking, the JAK/STAT reporter 6x2xDrafLuc was constructed by multimerisation of STAT92E binding sites. Specifically, a 165 bp blunted BamHI/Xbal fragment from the original p2xDrafSTAT(wt) (Kwon, E. J. et al., J Biol Chem 275, 19824-19830 (2000)) (a kind gift of M. Yamaguchi and M.A. Yoo) was inserted into the Smal cut p2xDrafSTAT(wt). The same fragment was amplified by PCR with NotI sites on both ends and inserted into compatible sites to yield the 3x2xDrafLuc reporter containing six STAT92E binding sites. These fragments were amplified again and the resulting 540 bp fragment was inserted into the Sacl cut 3x2xDrafLuc vector to generate the 6x2xDrafLuc reporter with an enhancer of approximately 1000 bp containing a total of 12 STAT92E binding sites. A second independent JAK/STAT pathway reporter, 4xsocsLuc, was generated by amplifying a 745 bp product from genomic DNA using the primers 5′-GTTAGGTACCGGGTCGCAGTATCGTTGGCG-3′ and 5′-CGMGGATCC CTGTCACTTCTCAGAAATCGGTC-3′. This was then cut with EcoRI/BamHI to give a 285 bp fragment, subcloned into pBS(KS+) (Stratagene) and re-excised with Asp718/BamHI. This 340 bp fragment, containing four predicted STAT92E binding sites (Karsten, P., Hader, S. & Zeidler, M., Mech Dev 117, 343. (2002)), was cloned into Asp718/BgIII sites of pGL3 vector (Promega).

The pAct-RL vector expressing Renilla luciferase from a constitutive reporter was generated by cloning a 974 bp fragment coding for Renilla luciferase from pRLSV40 (Invitrogen) into the BamHI/Xbal cut pP_{Ac5c}-PL vector (a kind gift from Dan Curtis). To generate the pAct-UpdGFP vector, a cDNA coding for Upd (Harrison, D. A., Binari, R., Nahreini, T. S., Gilman, M. & Perrimon, N., Embo J 14, 2857-65 (1995)) was fused in frame to EGFP via a BamHI site and inserted into the BamHI/Xbal cut pP_{Ac5c}-PL vector. A vector expressing the dominant gain-of-function allele Hop^{TumL} was cloned by inserting the open reading frame obtained from pUAS-hop^{TumL} (Harrison, D. A., Binari, R., Nahreini, T. S., Gilman, M. & Perrimon, N., Embo J 14, 2857-65 (1995)) into the NotI/Xbal cut pAc5.1A vector (Invitrogen). A pAc5.1-Sid-1 expression construct which was used to facilitate uptake of dsRNA was a gift of Craig Hunter (Feinberg, E. H. & Hunter, C. P., Science 301, 1545-7 (2003)).

To generate Ptp61F. expression constructs, cDNAs encoding Ptp61Fc (LP01280) and Ptp61Fa (RE01370) were obtained from the DrosophilaGenomics Resource Center (University of Indiana). cDNA clones were analysed by restriction analysis and end sequencing to confirm their integrity before subcloning into pAc5.1A and pUAST (Brand, A. H. & Perrimon, N., Development 118, 401-15 (1993)). For Ptp61Fc, the coding region of LP01280 was excised as an EcoRI/XhoI (partial digest) fragment of 1.8 kb and cloned into pUAST. Subsequently, the insert was re-excised with EcoRI/Xbal and cloned into pAc5.1A (Invitrogen). For Ptp61Fa, the coding region of RE01370 was cut out with EcoRI/Asp718(filled) and cloned into pAc5.1A cut EcoRI/Xbal(filled). The generate a pUAST construct, an EcoRI/Asp718 fragment was used.

To clone p[w⁺,UAS-dPIAS-GFP], the EST clone LD09022 was used as a template in conjunction with the oligos 5′-CATCGGATCCTGCAAAAAGGGG TCCAACGTACC GGAT-3′ and 5′-GGGGTACCAAAAATGGTGCATATGCTT CGA-3′ to amplify a region coding for 522 amino acids. The resulting product was sequenced, cut with Asp718/BamHI and subcloned into pBS-EGFP-B to generate an in frame C-terminal EGFP fusion protein. This gene was then subcloned as an Asp718/Xbal fragment into pUAST (Brand, A. H. & Perrimon, N., Development 118, 401-15 (1993)).

Multiple independent transgenic Drosophila stocks of each transformation vector construct were generated by microinjection of embryos using standard techniques (Spradling, A. C. & Rubin, G. M., Science 218, 341-347 (1982)).

### 1.2. Genome-Wide RNAi Screening

A genome-wide RNAi library based on PCR templates with an average length of 408 bp flanked by T7-promotor binding sites was generated by in vitro transcription (Boutros, M. et al., Science 303, 832-5 (2004)). Therefore, PCR fragments containing T7 promoter sequences on each end (Hild, M. et al., Genome Biol 5, R3 (2003)) were used as templates to generate 20,026 dsRNAs by in vitro transcription (Boutros, M. et al., Science 303, 832-5 (2004)). After DNAse I treatment, dsRNAs were purified by ethanol precipitation and individually quality controlled by gel electrophoresis. RNAs were diluted to a working stock concentration and aliquoted in ready-to-screen 384-well tissue culture plates (Greiner). Computational mapping predict that the 20,026 RNA fragments target >91% of all predicted genes in the Drosophila genome (Annotation 4.0) (Misra, S. et al., Genome Biol 3, RESEARCH0083-3 (2002)). Protocols and supplemental material can be found at http://www.dkfz-heidelberg.de/signaling/jak-pathway/. Complete primer and amplicon sequence information for double-stranded RNAs including calculation of predicted efficiency and off-target effects for the RNAi library is publicly accessible at http://rnai.dkfz.de.

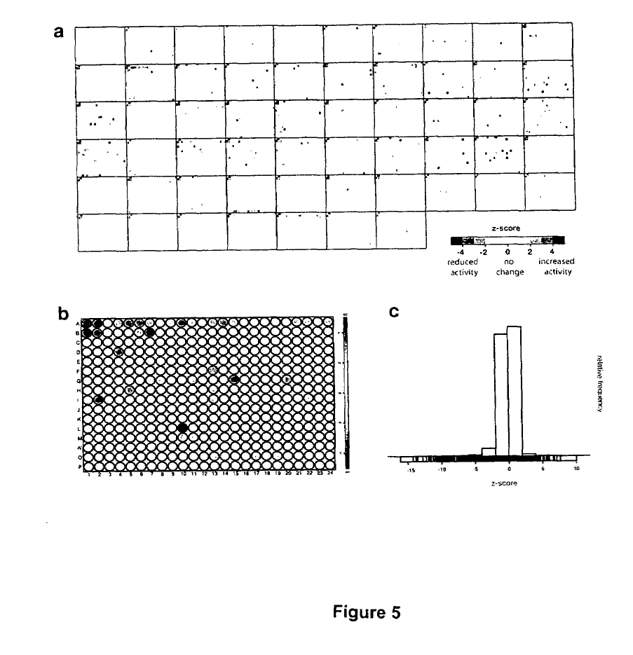

For screening experiments, Drosophila Kc₁₆₇ cells were maintained in Schneider's medium (Invitrogen) supplemented with 10% foetal bovine serum (PAA) and 100 μg/ml penicillin-streptomycin (Invitrogen). Cells were grown at 25° C. at subconfluent densities. The RNAi screening experiments were performed in white, polystyrene 384-well tissue culture plates (Greiner 781 073). A total of fiftyseven 384-well screening plates were loaded with an average of 75 nM (500 ng) dsRNA in 5 μl of 1 mM Tris pH 7. Kc₁₆₇ cells were transfected in batch in 6-well plates with 0.25 μg of the 6x2xDrafLuc JAK/STAT signalling reporter, 0.6 μg of pAct-UpdGFP expression vector, 0.25 μg pAc5.1-Sid-1 (to facilitate RNA uptake (Feinberg, E. H. & Hunter, C. P., Science 301, 1545-7 (2003))) and 0.025 μg of pAct-RL vector as a co-reporter. The total plasmid amount was normalised to 2 μg with a pAc5.1 plasmid (Invitrogen) and 5×10⁶ cells were transfected with Effectene (Qiagen). After 7 hours incubation at 25° C., batch transfected cells were resuspended in serum-free medium. Subsequently 15,000 cells in 20 μl were dispensed per dsRNA containing well using an automated liquid dispenser (MultiDrop, Thermo Labsystems). Cells were incubated for 45 min and 30 μl of serum-containing medium was added to each well. Cells were grown for 5 days to allow for protein depletion. Pathway activity was measured for using a luminescence assay for firefly and Renilla luciferase on a Mithras LB940 plate reader (Berthold Technologies). Luminescence of the Renilla luciferase was measured using a 490 nm filter set. Screens were performed in duplicate. Each plate contained dsRNA targeting stat92E, dome, hop and socs36E in A1, A2, B1, B2 which were used as positive controls (see also FIG. 5b). For retests, an independent JAK/STAT pathway reporter (4xsocsLuc) was used which contained a STAT-binding site from the endogenous JAK/STAT-pathway target socs36E (Karsten, P., Hader, S. & Zeidler, M., Mech Dev 117, 343. (2002)).

To identify candidate genes that significantly increase or decrease JAK/STAT signalling pathway activity, the raw luciferase results were normalised by median centering of each 384-well plate (separately by channel). Z-scores were calculated as the number standard deviation that a particular well differed from the median of the 384-well plate. To minimise false negatives, the inventors of the present invention applied a set of low-stringency criteria to generate a list of candidate genes to be used in specific retests. First, the inventors filtered dsRNA treatments with z-scores >2 for negative regulators or <−2 for positive regulators, respectively. Treatments that showed a high variability between duplicates were excluded. Further, RNAi experiments that showed z-scores of >2 or <−2 in the control channel were not selected for retesting. The inventors also filtered against previously identified cell viability modifiers that show a phenotype in cultured Drosophila cells (Boutros, M. et al., Science 303, 832-5 (2004)). The inventors also excluded genes that showed phenotypes in other screens. These filtering steps led to a final list of approximately 107 candidates that were selected for retesting. New dsRNA was re-synthesized as described above and repeat assays were performed in quadruplicate. 89 of the candidates were confirmed using a second JAK/STAT reporter assay (4xsocsLuc) employed to exclude reporter-specific artefacts. Data analysis and representation were performed using R and Bioconductor (Gentleman, R. C. et al., Genome Biol 5, R80 (2004)).

The predicted genes targeted by 91 dsRNAs were classified according to InterPro (Mulder, N. J. et al., Nucleic Acids Res 33 Database Issue, D201-5 (2005)) and GO (Harris, M. A. et al., Nucleic Acids Res 32, D258-61 (2004); Drysdale, R. A. et al., Nucleic Acids Res 33 Database Issue, D390-5 (2005)) and manual inspection was used to order genes into functional groups. Predicted proteins without InterPro domain or GO annotation were classified as “Unknown” although these sequences might encode structurally conserved proteins. To determine whether Drosophila proteins have homologues in other species, the inventors used BLASTP searches against the protein predictions from H. sapiens (NCBI build 35) with a cut-off of E<10⁻¹⁰. Databases were obtained from Ensembl (http://www.ensembl.org) (Clamp, M. et al., Nucleic Acids Res 31, 38-42 (2003)) and Flybase (hftp://www.flybase.org) (Drysdale, R. A. et al., Nucleic Acids Res 33 Database Issue, D390-5 (2005)). Reciprocal best BLASTP analysis was used to identify the human homologue of CG31132. CG31132 and human BRWD3 are classified as orthologous pairs by InParanoid (http://inparanoid.cgb.ki.se/).

### 1.3. Cell-Based Epistasis Experiments

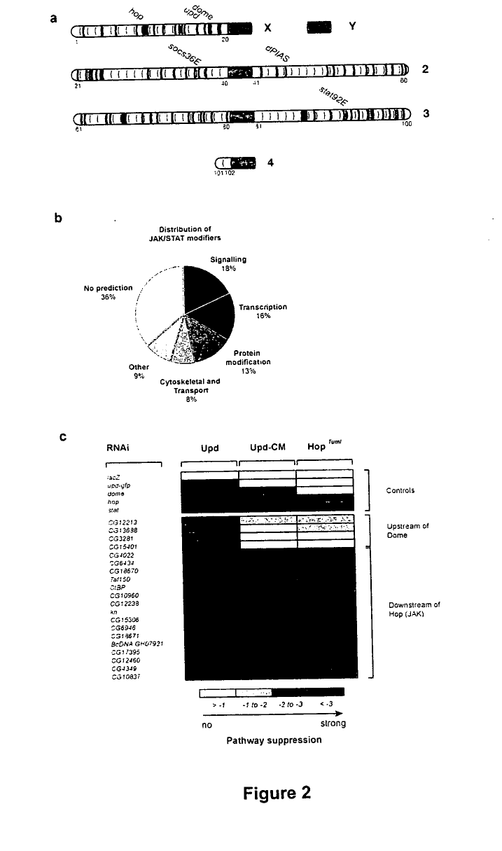

To undertake epistasis experiments, cells were transfected with vectors to stimulate pathway activity (see below) for 7 hours and 30,000 cells in 50 μl of serum-free medium were seeded into wells of clear bottom 96-well plates (Greiner), which contained 1.5 μg of the dsRNAs to be tested (listed in FIG. 2c). Following 1 hour incubation, 75 μl medium supplemented with 10% foetal bovine serum was added to the cells, plates were sealed and cells lysed after 5 days to measure luciferase activities.

Each dsRNA was tested for its ability to suppress pathway activity under three conditions: (1) in Upd-expressing cells (screening conditions), (2) in cells treated with Upd-conditioned medium (Upd-CM), and (3) in cells expressing the activated form of JAK, Hop^{Tuml} (Harrison, D. A., Binari, R., Nahreini, T. S., Gilman, M. & Perrimon, N., Embo J 14, 2857-65 (1995); Luo, H., Hanratty, W. P. & Dearolf, C. R., Embo J 14, 1412-20 (1995)). Specifically, for Upd overexpression 5×10⁶ Kcl₆₇ cells were transfected with 600 ng pAct-UpdGFP, 500 ng 6x2xDrafLuc reporter, 250 ng pAc5.1-Sid-1, 25 ng pAct-RL and pAc5.1 to a total of 2 μg DNA. For Hop^{TumL} overexpression, 5×10⁶ Kc₁₆₇ cells were transfected with 200 ng pAct-hop^{TumL}, 500 ng 6x2xDrafLuc reporter, 250 ng pAc5.1-Sid-1, 25 ng pAct-RL and pAc5.1 to a total of 2 μg DNA. To analyse processes upstream of Upd, two batches of cells were transfected separately to generate ‘responder’ and ‘Upd-producer’ cells. The ‘responder’ cells were batch transfected with 500 ng 6x2xDrafLuc reporter, 250 ng pAc5.1-Sid-1, 25 ng pAct-RL and pAc5.1 to a total of 2 μg plasmid DNA and subsequently seeded into 96-well plates containing the respective dsRNAs as described above. The ‘Upd-producing’ cells were transfected with 2 μg pAct-UpdGFP and cultured in 10 cm dishes (Falcon). Three days after transfection, cells were treated with 50 μg/ml Heparin (Sigma). After 24 hours, the supernatant was harvested, cleared by centrifugation and passed through a 0.2 μm filter (Millipore). 50 μl of this Upd-conditioned medium were then used to stimulate pathway activity in the ‘responder’ cells for 24 hours. Control medium from untransfected Heparin treated cells did not elicit pathway activity (not shown).

Experiments were performed in eight replicates and repeated at least twice. Reporter activity in the firefly luciferase channel was divided by the Renilla luciferase channel to normalise for cell number. Z-scores were calculated as the multiples of the standard deviation that a specific RNAi treatment differed from cells treated with lacZ dsRNA as negative controls. Z-scores were subsequently transformed into a false-colour representations as depicted in FIG. 2c.

RNA controls as shown in FIG. 2c were in vitro transcribed from PCR templates generated using the following gene-specific primer sequences: 5T7lacZ: GAATAATACGACTCACTATAGGGAGACAGTGGCGTCTGGCGGAAAA (SEQ ID NO. 448), 37lacZ: GMTTMTACGACTCACTATAGGGAGATCCGAGCC AGTTTACCCGCT (SEQ ID NO. 449), 5T7gfp: TMTACGACTCACTATAGGACGGC CGCCATTMCMGCAAAAG (SEQ ID NO. 450) and 3T7gfp: TAATACGACTCACT ATAGGCTGGGCGGAGCGGATGATG (SEQ ID NO. 451). Note that the gfp dsRNA was used to target the Upd-GFP transgene and leads to a loss-of pathway activity. lacZ dsRNA was used as a negative control.

For epistasis analysis of the putative negative regulator ptp61F, cells were batch transfected with reporter and Upd inducer as described above. Subsequently, these cells were treated with 1.5 μg of dsRNA targeting the ptp61F transcript and 1.5 μg of dsRNA against lacZ, dome, hop or stat92E. In parallel, cells from the same transfection batch were treated with lacZ, dome, hop or stat92E dsRNAs alone. After normalisation, the values of experiments with control dsRNA alone were set to one. To examine the JAK/STAT phenotype of ptp61F in cells, 5×10⁶ Kc₁₆₇ cells were transfected with 0.6 μg pAct-UpdGFP, 0.5 μg 6x2xDrafLuc reporter, 0.25 μg pAc5.1-Sid-1, 0.025 μg pAct-RL and pAc5.1 to a total of 2 μg DNA. To assess the effects of the different Ptp61F splice forms, cells were transfected as described before with additional 0.5 μg of pAct-Ptp61Fa, pAct-Ptp61Fc or vector control, respectively. JAK/STAT pathway activation was expressed in relation to control cells.

### 1.4. Genetics

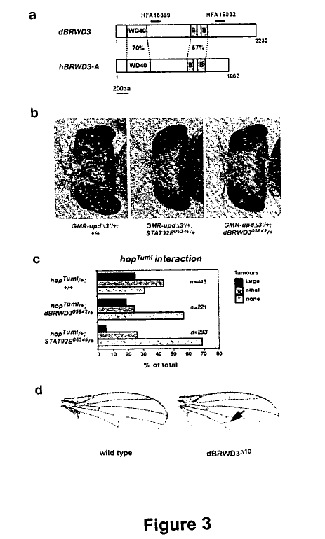

A P-element insertion termed I(3)05842 (Spradling, A. C. et al., Genetics 153, 135-77 (1999)) was identified in the fourth intron of dBRWD3/CG31132 as part of a Flybase search (Drysdale, R. A. et al., Nucleic Acids Res 33 Database Issue, D390-5 (2005)). A I(3)05842 stock was obtained from the Bloomington stock centre (University of Indiana). The P-element insertion I(3)05842 is homozygous lethal and fails to complement the Df(3R)crb874 and Df(3R)crb87-5 deficiencies. Twenty three independent stocks in which the ry⁺ marker present in the P[ry⁺,PZ] insertion had been lost following a cross to a transposase source were established. Of these, seven were viable revertants (30%) and include two stocks with the wing vein phenotype (FIG. 3d), two are semi-lethal with occasional escapers and the remainder were lethal.

For genetic interaction assays, females of the stock y,w,hop^{Tuml}/FM7; P [w⁺,cg-Gal4.A]2 (Harrison, D. A., Binari, R., Nahreini, T. S., Gilman, M. & Perrimon, N., Embo J 14, 2857-65 (1995)) were crossed to wild type controls (OreR and w¹¹¹⁸) and mutations in stat92E and I(3)05842. The haemocyte specific Gal4 driver line P[w⁺,cg-Gal4.A]2 allowed specific UAS insertions to be tested for their potential influence on tumour formation. Transgenic animals expressing UAS-EGFP or UAS-β-galactosidase were used as negative controls while UAS-dPIAS-EGFP served as a positive control (Betz, A., Lampen, N., Martinek, S., Young, M. W. & Darnell, J. E., Jr., Proc Natl Acad Sci USA 98, 9563-8 (2001)) (see Table 3).

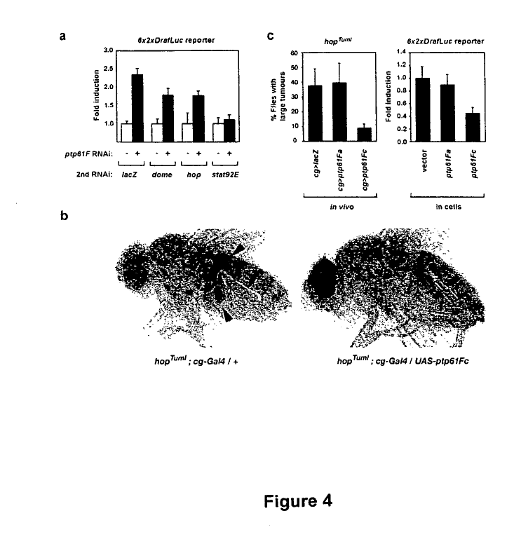

Crosses were incubated at 25° C. and adult females heterozygous for the hop^{Tuml} chromosome were scored within 24 hours of eclosion for the presence of tumours classified as small (one or two small melanotic spots as shown in FIG. 4b [right]) or large (one or more large melanised growths or more than three small spots; FIG. 4b [left]). Survival rates for hop^{Tuml} females appear to be independent of tumour frequency at the time point counted (not shown). Assays were repeated at least twice for each genotype and a representative example from one experiment is shown (FIG. 4b).

Genetic interaction with P[w⁺,GMR-updΔ3′]′19 was undertaken as described in Genetics 165, 1149-66 ((2003), Bach, E. A., Vincent, S., Zeidler, M. P. & Perrimon, N.) using OreR and STAT92E⁰⁶³⁴⁶ as negative and positive controls, respectively. Suppression of P[w⁺,GMR-updΔ3′]′19 induced eye overgrowth by dBRWD3⁰⁵⁸⁴² was observed in multiple independent experiments in a majority of individuals of the appropriate genotype. Drosophila heads were photographed using a Zeiss STEMI 2000-C binocular microscope and Axiocam camera.

### 2. Results and Discussion

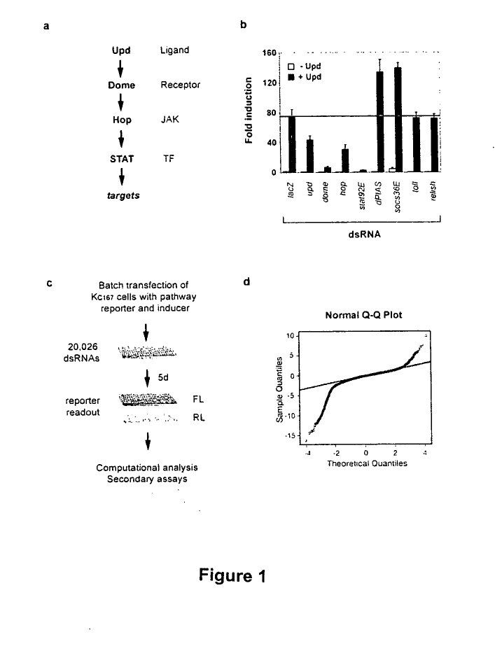

Developmental genetic screens in Drosophila have identified multiple JAK/STAT pathway components on the basis of their segmentation phenotype (Binari, R. & Perrimon, N., Genes Dev 8, 300-12. (1994); Harrison, D. A., McCoon, P. E., Binari, R., Gilman, M. & Perrimon, N., Genes Dev 12, 3252-63. (1998); Hou, X. S., Melnick, M. B. & Perrimon, N., Cell 84, 411-9 (1996)) and subsequent analysis of the pathway has characterised evolutionarily conserved roles during immune responses, haematopoiesis and cellular proliferation (Lagueux, M., Perrodou, E., Levashina, E. A., Capovilla, M. & Hoffmann, J. A., Proc Natl Acad Sci USA 97, 11427-32. (2000); Boutros, M., Agaisse, H. & Perrimon, N., Dev Cell 3, 711-22. (2002); Meister, M. & Lagueux, M., Cell Microbiol 5, 573-580 (2003); Mukherjee, T., Castelli-Gair Hombria, J. & Zeidler, M. P., Oncogene in press (2005)). The JAK/STAT signalling cascade in Drosophila is comprised of the extracellular ligand Unpaired (Upd) (Harrison, D. A., McCoon, P. E., Binari, R., Gilman, M. & Perrimon, N., Genes Dev 12, 3252-63. (1998)), a trans-membrane receptor with homology to the IL6 receptor family termed Domeless (Dome) (Brown, S., Hu, N. & Castelli-Gair Hombria, J., Curr Biol 11, 1700-5. (2001)), a single Janus tyrosine kinase (JAK) called Hopscotch (Hop) (Binari, R. & Perrimon, N., Genes Dev 8, 300-12. (1994)) and the STAT92E transcription factor (Hou, X. S., Melnick, M. B. & Perrimon, N., Cell 84, 411-9 (1996); Yan, R., Small, S., Desplan, C., Dearolf, C. R. & Darnell, J. E., Jr., Cell 84, 421-30 (1996)) (FIG. 1a). Known regulators of JAK/STAT signalling including a family of SOCS-like genes (Callus, B. A. & Mathey-Prevot, B.; Oncogene 21, 4812-4821 (2002); Karsten, P., Hader, S. & Zeidler, M. P., Mech Dev 117, 343-6 (2002)), dPIAS/Su(var)2-10 (Betz, A., Lampen, N., Martinek, S., Young, M. W. & Darnell, J. E., Jr., Proc Natl Acad Sci USA 98, 9563-8 (2001)) and STAM (Mesilaty-Gross, S., Reich, A., Motro, B. & Wides, R., Gene 231, 173-86 (1999)) are functionally conserved and were identified based on their homology to components originally characterised in mammalian cell culture studies (Hombria, J. C. & Brown, S., Curr Biol 12, R569-75 (2002)). Although successful in identifying the pathway members Upd, Dome, Hop and STAT92E, it is probable that forward genetic approaches have missed components possibly due to non-saturating mutagenesis, genetic redundancy or phenotypic pleiotropy (Nagy, A., Perrimon, N., Sandmeyer, S. & Plasterk, R., Nat Genet 33 Suppl, 276-84 (2003)).

In order to identify novel pathway components and circumvent limitations of classical genetic screens, the inventors of the present invention have undertaken a genome-wide RNA interference (RNAi) screen, a powerful technique for the identification of new components of diverse cellular pathways (Kamath, R. S. et al., Nature 421, 231-7 (2003); Kittler, R. et al., Nature 432, 1036-40 (2004); Berns, K. et al., Nature 428, 431-7 (2004); Paddison, P. J. et al., Nature 428, 427-31 (2004); Boutros, M. et al., Science 303, 832-5 (2004)). To this end, the inventors devised a quantitative assay for JAK/STAT signalling activity in cultured Drosophila cells by multimerising a STAT92E-binding site from the Draf promotor (Kwon, E. J. et al., J Biol Chem 275, 19824-19830 (2000)) to generate the 6x2xDrafLuc firefly luciferase reporter. Given the role for JAK/STAT signalling in haematopoiesis (Meister, M. & Lagueux, M., Cell Microbiol 5, 573-580 (2003)), the inventors used Drosophila hemocyte-like Kc₁₆₇ cells due to their endogenous ability to respond to pathway activation (FIG. 1b). On transfection of the 6x2xDrafLuc reporter and a plasmid to constitutively express the ligand Upd, a robust induction of the reporter gene activity was observed (FIG. 1b). The inventors first examined whether depletion of known pathway components by RNAi (Clemens, J. C. et al., Proc Natl Acad Sci U S A 97, 6499-6503 (2000)) modifies JAK/STAT signalling activity in Kc₁₆₇ cells. The inventors assessed the effect of double-stranded (ds) RNAs targeting the mRNA of the genes dome, stat92E and hop, as well as dsRNAs directed against the negative regulators socs36E and dPIAS. As shown in FIG. 1b, knock down of JAK/STAT components results in significant changes in reporter activity while reporter activity in uninduced cells remains at low levels (FIG. 1b).

The inventors then set out to systematically identify genes required for JAK/STAT signalling by generating a library of 20,026 dsRNAs targeting 91% of the predicted transcripts in the Drosophila genome. Using this library the inventors performed duplicate genome-wide screens as outlined in FIGS. 1c and 5. After computational analysis (FIG. 1d), dsRNAs targeting candidates were resynthesised and assayed with an independent reporter, derived from the promoter of the pathway target gene socs36E (Karsten, P., Hader, S. & Zeidler, M. P., Mech Dev 117, 343-6 (2002)) to exclude reporter specific artefacts. These approaches confirmed the identification of 71 dsRNAs which decrease pathway activity (targeting putative positive regulators) and 19 dsRNAs which increase pathway activity (putative negative regulators) (see Table 1). While most modifiers are distributed throughout the genome (FIG. 2a), the X chromosome is devoid of negative regulators, a finding which may be linked to the role of the pathway in Drosophila sex determination (Sefton, L., Timmer, J. R., Zhang, Y., Beranger, F. & Cline, T. W., Nature 405, 970-3 (2000)).

Based on InterPro and GO annotations, pathway modifiers were classified according to their predicted functions. Signalling factors, enzymes mediating post-translational protein modifications and transcription factors cumulatively represent 47% of the genes identified (FIG. 2b). Furthermore, 74% of the identified loci possess human homologues (E-value <10⁻¹⁰), 33% of which have been implicated in human disease (Tables 5 and 6). Examples of genes identified in the screen include CG11501 encoding a putatively secreted negative regulator of JAK/STAT signalling previously demonstrated to be a pathway target gene (Boutros, M., Agaisse, H. & Perrimon, N., Dev Cell 3, 711-22. (2002)), enok/CG11290 encoding an acetyl-transferase and the tumor suppressor protein 101/CG9712 gene which encodes a ubiquitin conjugating enzyme. The molecular role of these genes in the regulation of JAK/STAT signalling remains to be determined.

A genetic technique to characterise signalling molecules is the determination of their epistatic relationship with respect to defined pathway components. The inventors therefore performed cell-based epistatic assays to determine the pathway response to Upd expression, Upd conditioned medium or expression of the constitutively active JAK allele hop^{Tuml} (Harrison, D. A., McCoon, P. E., Binari, R., Gilman, M. & Perrimon, N., Genes Dev 12, 3252-63. (1998); Sefton, L., Timmer, J. R., Zhang, Y., Beranger, F. & Cline, T. W., Nature 405, 970-3 (2000)) while simultaneously targeting a subset of positive regulators. In this way, dsRNA-inactivated genes required upstream in the pathway can be characterised on the basis of their rescue by pathway activation further downstream (FIG. 2c). For example, while depletion of the interferon-related protein encoded by CG15401 results in down-regulation of signalling stimulated by Upd expression, stimulation by Upd conditioned medium or hop^{Tuml} is unaffected (FIG. 2c). This suggests that CG15401 is required for the production and/or activity of the Upd ligand. Conversely, loss of pathway activity resulting from the knock down of CG18670 and CG6400 (now annotated as one gene termed CG31132) cannot be rescued by any form of pathway stimulus implying a function downstream of JAK (FIG. 2c). Although this analysis suggests a role for multiple genes upstream of Dome, this classification is based on the lack of interaction observed under the differing experimental conditions and the molecular basis of these results remains to be confirmed.

In order to confirm the function of candidate genes in vivo, the inventors tested examples of both positive and negative regulators of the JAK/STAT signalling pathway. One positive regulator mentioned above is CG31132 which encodes a 2232 amino acid WD40 and bromo-domain containing protein homologous to human BRDW3 (FIG. 3a). BRDW3 is a functionally uncharacterised locus recently identified at the break point of t(X;11) (q13;q23) translocations derived from multiple B-cell chronic lymphocytic leukaemia (B-CLL) patients (Kalla, C. et al., Genes Chromosomes Cancer 42, 128-43 (2005)). In the screen, reduction of pathway activity was observed for two independent dsRNAs present in the library that target different regions of the transcript (FIG. 3a).

A previously uncharacterised mutagenic P-element inserted in the fourth intron of CG31132 (henceforth termed dBRDW3⁰⁵⁸⁴²) has been deposited in public stock collections as part of the Drosophila genome project and remobilisation of this transposon indicates that the insertion is responsible for late embryonic lethality. The inventors therefore tested for genetic interactions between dBRDW3 and JAK/STAT signalling by crossing the dBRDW3⁰⁵⁸⁴² allele to GMR-updΔ3′ (Bach, E. A., Vincent, S., Zeidler, M. P. & Perrimon, N., Genetics 165, 1149-66 (2003)). The GMR-upd□3′ transgene ectopically misexpress Upd during eye development resulting in cellular overproliferation and an enlarged adult eye (FIG. 3b (left)). Furthermore, removal of one copy of stat92E significantly suppresses eye overgrowth (FIG. 3b (middle)) due to a reduction in the potency of JAK/STAT signalling (Bach, E. A., Vincent, S., Zeidler, M. P. & Perrimon, N., Genetics 165, 1149-66 (2003)). Removal of a single copy of dBRDW3 was also able to suppress the GMR-updΔ3′ phenotype (FIG. 3b (right)) as expected for a positive regulator of JAK/STAT signalling. In addition, a chromosomal deficiency removing the region has also been independently identified as a suppressor of GMR-updΔ3′ (Bach, E. A., Vincent, S., Zeidler, M. P. & Perrimon, N., Genetics 165, 1149-66 (2003)).

One phenotypic consequence of constitutive JAK/STAT activation caused by the gain-of-function JAK allele hop^{Tuml} is the overproliferation of haemocytes and the frequent formation of melanotic tumours, a phenotype previously described as a Drosophila model for leukaemia (Luo, H., Hanratty, W. P. & Dearolf, C. R., Embo J 14, 1412-20 (1995); Harrison, D. A., Binari, R., Nahreini, T. S., Gilman, M. & Perrimon, N., Embo J 14, 2857-65 (1995)). The inventors found that the removal of one copy of dBRWD3 is sufficient to reduce the size and the frequency of hop^{Tuml} induced melanotic tumours (FIG. 3c and Table 3). Moreover, homozygous escapers of a putative hypomorphic allele of dBRWD3, generated by excision of the original P-element, frequently develop ectopic wing vein material (FIG. 3d) reminiscent of the weak loss-of-function stat92E^{HJ} allele (Yan, R., Luo, H., Darnell, J. E., Jr. & Dearolf, C. R., Proc Natl Acad Sci USA 93, 5842-7 (1996)). Taken together, these experiments suggest a role for dBRWD3 in JAK/STAT signalling.

As a second example the inventors analysed the ptp61F gene which encodes a protein tyrosine phosphatase. dsRNA knocking down all mRNA splice forms transcribed from this locus leads to an increase in JAK/STAT signalling activity. The inventors performed epistasis analysis in which the inventors removed known pathway components and tested for the effect of simultaneously targeting ptp61F. Double RNAi against ptp61F together with lacZ, dome or hop results in pathway stimulation (FIG. 4a). However, simultaneous removal of ptp61F and stat92E is sufficient to prevent signalling (FIG. 4a). Loss of this phosphatase therefore results in the stimulation of STAT92E activity even in the absence of upstream components indicating that Ptp61F negatively regulates the pathway downstream of JAK. The inventors next asked whether Ptp61F also interferes with JAK/STAT signalling in vivo by using the cg-Gal4 driver to misexpress ptp61F in blood cells of hop^{Tuml} mutants. Misexpression of Ptp61Fc in a hop^{Tuml} mutant background resulted in a suppression of melanotic tumour formation with the average frequency of large tumours reduced by approximately 4 fold, an effect also observed following the misexpression of the known negative regulator dPIAS (Betz, A., Lampen, N., Martinek, S., Young, M. W. & Darnell, J. E., Jr., Proc Natl Acad Sci USA 98, 9563-8 (2001)) (FIG. 4b and Table 3). Alternative splicing of ptp61F leads to nuclear and cytoplasmic protein forms which both contain the same phosphatase domain (McLaughlin, S. & Dixon, J. E., J Biol Chem 268, 6839-42 (1993)). However, the tumour suppressor phenotype is only observed with nuclear Ptp61Fc (FIG. 4c), an effect that is reproduced by over-expression of the nuclear localised protein in cell culture (FIG. 4c). These results are consistent with our identification of ptp61F as a negative regulator of pathway activity and suggest that it may function by targeting phosphorylated, nuclear localised STAT92E for deactivation.

Aberrant JAK/STAT signalling has been implicated in multiple human malignancies and its components have been proposed as molecular targets for the development of therapeutic compounds (O'Shea, J. J., Pesu, M., Borie, D.C. & Changelian, P. S., Nat Rev Drug Discov 3, 555-64 (2004)). The genome-wide screen presented here identified known and previously unknown genes and the inventors have characterised their likely level of interaction with defined pathway components using cell-based epistasis analysis. Of the 89 JAK/STAT modifiers identified, many have human homologues that remain to be characterised. The inventors have here performed an analysis of two examples in vivo and demonstrate their roles in regulating the pathway during development and tumour genesis in Drosophila. One of these is a homologue of human BRWD3, a gene recently identified at the break-point of a translocation isolated from multiple B-CLL patients (Kalla, C. et al., Genes Chromosomes Cancer 42, 128-43 (2005)). Given our functional analysis of dBRWD3 and the known roles for JAK/STAT signalling during normal haematopoiesis, it is possible that a breakdown in BRWD3 mediated STAT regulation may represent a key molecular mechanism leading to the development of B-CLL. Thus, comprehensive reverse genetic surveys for signalling pathway components using Drosophilaas a model organism represent a potentially powerful approach with which insights relevant to human disease can be obtained.

### Example 2

Novel components regulating the JAK/STAT pathway in Drosophilamelanogaster have been previously been identified using a robust STAT92E responsive reporter assay in combination with genome-wide RNAi (Müller, P., Kuttenkeuler, D., Gesellchen, V. Zeidler, M.P. and Boutros M. (2005) “Identification of JAK/STAT signalling components by genome-wide RNAi” Nature 436 871-875). Having identified the essential components in Drosophila, a second crucial step is the identification of human functional orthologs. Given that many of the potential human orthologs have been implicated in human disease, these proteins, and the mRNAs that encode them, may represent targets for therapeutic interventions by small molecules or RNAi based approaches. Using a HeLa cell model we have assayed the activity of endogenous STAT1 and STAT3 following treatment with siRNA targeting potential pathway modulating genes. Assays of hSTAT phosphorylation state and the expression levels of their targets, have identified 27 human genes, which function as modulators of human JAK/STAT signal transduction. These have been ranked on the basis of potential significance and are listed in Table 7 together with the human diseases they have previously been associated with.

### Results

Compared to Drosophila, the JAK/STAT pathway in mammalians is much more complex in that multiple paralogs exist for the pathway ligand, receptor, JAK and STAT. As an initial approach towards identifying regulators of human JAK/STAT signaling, we have analyzed phenotypes caused by siRNA-mediated knockdown of candidate pathway modifiers in human cells. Human genes for this analysis were selected based on their homology to Drosophila JAK/STAT pathway regulators previously identified (Müller et al. 2005). Homology prediction by a variety of methods yielded 73 candidates homologous to 56 Drosophila genes. Pools of 4 siRNAs per candidate (Dharmacon SMARTpools) were used to ensure the efficiency and specificity of knockdown. As an easily tractable model, we have used human cancer-derived HeLa cells which express multiple STATs and which respond to stimulation by a variety of cytokine ligands (Ehret G.B., Reichenbach P., Schindler U., Horvath C.M., Fritz S., Nabholz M., Bucher P. (2001) “DNA binding specificity of different STAT proteins. Comparison of in vitro specificity with natural target sites” J Biol Chem 276 6675-6688).

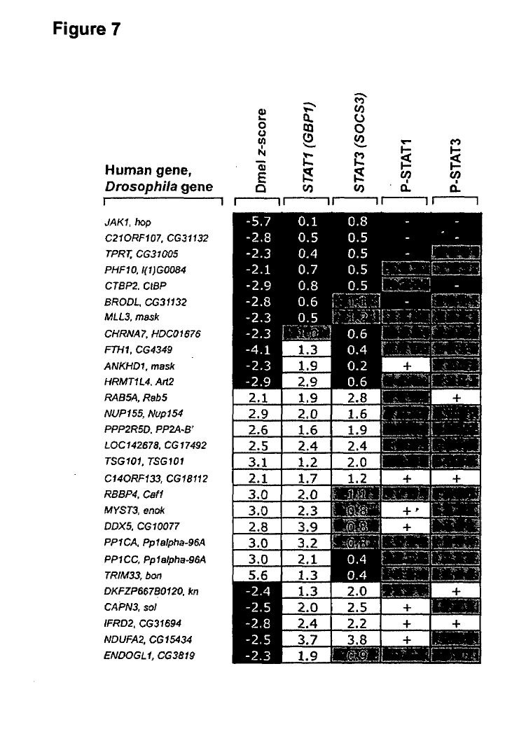

Two approaches have been used to determine the activity of STAT1 and STAT3 in the HeLa cell system tested. Firstly, the levels of tyrosine-701-phosphorylated STAT1 (pSTAT1) and tyrosine-705-phosphorylated STAT3 (pSTAT3) were determined in HeLa cell lysates that had been stimulated with human Interferon gamma (INFγ) or Oncostatin M (OSM) for 15 min, respectively. These cells had previously been treated with siRNA targeting either controls or the putative pathway interactors for 72hs. After determination of the overall level of STAT1/3, the western blots were stripped and re-probed with pSTAT1 and pSTAT3 antibodies and with antibodies to determine β-ACTIN levels as a normalization control. The relative levels of pSTAT1/3 versus STAT1/3 were assessed with regard to the overall level of β-ACTIN detected and a call made representing either an increase in PSTAT levels (+), a decrease in PSTAT (−) or no change (FIG. 7 column 4 & 5).

As a second independent approach to determine the level of STAT1 and STAT3 activity, the expression levels of the previously characterized pathway target genes GBPI (a STAT1 target) and SOCS3 (a STAT3 target) were determined 6 hrs after stimulation of HeLa cells with INFy and OSM, respectively. As before, cells had previously been treated with siRNA targeting either controls or putative pathway regulators for 72 hrs. Target gene levels were determined using branched DNA technology (QuantiGene, Panomics) and normalized to the level of β-actin mRNA. Results from duplicate assays are expressed as fold changes in target gene expression levels relative to cells treated with control siRNA. Statistically significant changes in response (p<0.05) are shown in black (decrease in expression level) or white (increase in expression) (FIG. 7 column 2 & 3, Table 8 column 5 & 6). In this table the scores relating to hSTAT1 and hSTAT3 target genes are expressed such that 1 is the expression level induced by pathway ligands following treatment with a control siRNA. Numbers below 1 therefore indicate a reduction in expression while scored above 1 represent an increase. Scores for Drosophila STAT92E are expressed as z-scores—a measure of statistical significance in which significant suppression of activity is represented by numbers <−2.0 while significant enhancement is represented by values >+2.0. Statistically significant changes are indicated by the change in colour of the boxes shown in FIG. 7. Note that only genes which interact via at least one assay are shown and other human homologues of interacting Drosophila genes not listed did not show any interaction with the STAT1 or STAT3 assays used.

Analysis of these two independent data sets, in conjunction with the scores originally obtained for the Drosophila orthologs (FIG. 7 column 1 and Table 8, column 4) has identified positively acting factors that are required for both STAT1 & 3 as well as factors that are required specifically by only STAT1 or STAT3. In addition negatively acting factors acting on either or both STATs have been found. Finally, some factors act positively for one STAT and negatively for another (this may be a result of redundancy within the pathway) while others act as positive regulators in Drosophila but as negative regulators in human cells. This analysis has lead to the compilation of a list of human genes playing a role in the regulation of human JAK/STAT signaling (Table 7). These genes have been ranked by order of interest (highest at the top; Table 7) as judged by criteria such as involvement in human disease, predicted sub-cellular localization and strength of interaction.

TABLE 1
JAK/STAT phenotypes by RNAi

| | | | | | | | |
|---|---|---|---|---|---|---|---|
| | | z-score | z-score | | | | |
| | | [6 × 2 × Draf- | [4 × SOCS- | Functional group assignment (based on GO | Interpro 8.0 | | |
| Gene name | dsRNA ID | luc] | luc] | and Interpro evidence) | evidence | GO Evidence | SEQ ID NO. |

Positive Regulators

| | | | | | | | |
|---|---|---|---|---|---|---|---|
| Art2 | HFA00627 | −2.9 | −3.2 | Protein modifying enzymes/Metabolism | IPR000051 | GO: 0016274; protein-arginine N-methyltransferase activity | SEQ ID NO. 175 |
| asf1 | HFA11324 | −2.3 | −2.5 | Others | IPR008967 | GO: 0003682; chromatin binding | SEQ ID NO. 176 |
| bin3 | HFA04919 | −3.1 | −3.3 | Unknown | IPR000051 | na; na | SEQ ID NO. 177 |
| CG10007 | HFA14173 | −3.2 | −2.9 | Unknown | noIPR | na; na | SEQ ID NO. 178 |
| CG10730 | HFA02102 | −2.1 | −2.3 | Unknown | IPR004245 | na; na | SEQ ID NO. 179 |
| CG10960 | HFA09807 | −2.0 | −2.1 | Protein modifying enzymes/Metabolism | IPR005829 | GO: 0005355; glucose transporter activity | SEQ ID NO. 180 |
| CG11307 | HFA11648 | −2.3 | −2.4 | Unknown | noIPR | GO: 0016757; transferase activity | SEQ ID NO. 181 |
| CG11696 | HFA19417 | −2.0 | −2.3 | Transcription regulators | IPR007087 | GO: 0003677; DNA binding | SEQ ID NO. 182 |
| CG12213 | HFA14478 | −3.3 | −3.2 | Unknown | IPR009053 | na; na | SEQ ID NO. 183 |
| CG12460 | HFA20970 | −3.3 | −3.4 | Transcription regulators | IPR000504 | GO: 0030528; transcription regulator activity | SEQ ID NO. 184 |
| CG12479 | HFA19459 | −2.3 | −2.4 | Unknown | IPR007512 | na; na | SEQ ID NO. 185 |
| CG13243 | HFA01920 | −2.7 | −2.6 | Unknown | IPR003117 | na; na | SEQ ID NO. 186 |
| CG13473 | HFA10017 | −2.4 | −2.1 | Cytoskeleton and Transport | IPR006662 | GO: 0005489; electron transporter activity | SEQ ID NO. 187 |
| CG14434 | HFA17927 | −2.0 | −2.3 | Unknown | IPR008173 | na; na | SEQ ID NO. 188 |
| CG15306 | HFA17993 | −3.3 | −3.1 | Signal transduction | IPR001715 | GO: 0005102; receptor binding | SEQ ID NO. 189 |
| CG15418 | HFA00432 | −2.1 | −2.1 | Protein modifying enzymes/Metabolism | IPR002223 | GO: 0004866; endopeptidase inhibitor activity | SEQ ID NO. 190 |
| CG15434 | HFA00449 | −2.5 | −2.9 | Protein modifying enzymes/Metabolism | IPR007741 | GO: 0003954; NADH dehydrogenase activity | SEQ ID NO. 191 |
| CG15555 | HFA15093 | −2.3 | −2.6 | Others | IPR001873 | GO: 0015268; alpha-type channel activity | SEQ ID NO. 192 |
| CG15784 | HFA18090 | −2.4 | −2.6 | Unknown | IPR009072 | na; na | SEQ ID NO. 193 |
| CG16903 | HFA18561 | −2.8 | −2.8 | Transcription regulators | IPR011028 | GO: 0016251; general RNA polymerase II transcription factor activity | SEQ ID NO. 194 |
| CG17179 | HFA10258 | −2.1 | −2.8 | Unknown | IPR001680 | na; na | SEQ ID NO. 195 |
| CG18160 | HFA21006 | −3.1 | −2.4 | Unknown | noIPR | na; na | SEQ ID NO. 196 |
| CG30069 | HFA06272 | −2.9 | −2.2 | Protein modifying enzymes/Metabolism | noIPR | GO: 0016491; oxidoreductase activity | SEQ ID NO. 197 |
| CG3058 | HFA00563 | −3.4 | −3.5 | Cytoskeleton and Transport | IPR006663 | GO: 0005489; electron transporter activity | SEQ ID NO. 198 |
| CG31005 | HFA15507 | −2.3 | −3.0 | Protein modifying enzymes/Metabolism | IPR000092 | GO: 0000010; trans-hexaprenyltranstransferase activity | SEQ ID NO. 199 |
| CG31132 | HFA16032 | −2.8 | −3.5 | Unknown | IPR001487 | na; na | SEQ ID NO. 200 |
| CG31132 | HFA15369 | −2.3 | −3.6 | Unknown | IPR001487 | na; na | SEQ ID NO. 201 |
| CG31358 | HFA15235 | −2.0 | −2.2 | Cytoskeleton and Transport | IPR001972 | GO: 0005200; structural constituent of cytoskeleton | SEQ ID NO. 202 |
| CG31694 | HFA00415 | −2.8 | −2.7 | Signal transduction | IPR006921 | GO: 0005102; receptor binding | SEQ ID NO. 203 |
| CG32406 | HFA09966 | −2.1 | −2.2 | Signal transduction | IPR000980 | na; na | SEQ ID NO. 204 |
| CG32573 | HFA19906 | −3.1 | −2.9 | Unknown | IPR000719 | na; na | SEQ ID NO. 205 |
| CG3281 | HFA15470 | −3.1 | −3.0 | Transcription regulators | IPR007087 | GO: 0030528; transcription regulator activity | SEQ ID NO. 206 |
| CG3819 | HFA10378 | −2.3 | −2.3 | Unknown | IPR001604 | na; na | SEQ ID NO. 207 |
| CG4022 | HFA10395 | −3.4 | −3.7 | Unknown | noIPR | na; na | SEQ ID NO. 208 |
| CG40351 | HFA20930 | −2.6 | −2.7 | Transcription regulators | IPR001214 | GO: 0030528; transcription regulator activity | SEQ ID NO. 209 |
| CG4349 | HFA19892 | −4.1 | −2.1 | Others | IPR009040 | GO: 0008199; ferric iron binding | SEQ ID NO. 210 |
| CG4446 | HFA10420 | −2.7 | −2.7 | Protein modifying enzymes/Metabolism | IPR004625 | GO: 0008478; pyridoxal kinase activity | SEQ ID NO. 211 |
| CG4653 | HFA19909 | −3.2 | −3.0 | Protein modifying enzymes/Metabolism | IPR001254 | GO: 0004263; chymotrypsin activity | SEQ ID NO. 212 |
| CG4781 | HFA04488 | −2.5 | −2.5 | Unknown | IPR003591 | na; na | SEQ ID NO. 213 |
| CG6422 | HFA16036 | −3.3 | −3.2 | Unknown | IPR007275 | na; na | SEQ ID NO. 214 |
| CG6434 | HFA10635 | −2.8 | −2.8 | Unknown | IPR001680 | na; na | SEQ ID NO. 215 |
| CG6946 | HFA16145 | −2.3 | −2.9 | RNA processing and Translation | IPR000504 | GO: 0003723; RNA binding | SEQ ID NO. 216 |
| CG7635 | HFA20054 | −2.9 | −2.8 | Cytoskeleton and Transport | IPR001972 | GO: 0005200; structural constituent of cytoskeleton | SEQ ID NO. 217 |
| CG8108 | HFA09675 | −2.7 | −2.7 | Transcription regulators | IPR007087 | GO: 0003676; nucleic acid binding | SEQ ID NO. 218 |
| CG9086 | HFA20148 | −2.8 | −2.9 | Signal transduction | IPR009030 | GO: 0005057; receptor signaling protein activity | SEQ ID NO. 219 |
| CkIIalpha | HFA11946 | −2.1 | −2.5 | Signal transduction | IPR000719 | GO: 0004702; receptor signaling protein serine/threonine kinase activity | SEQ ID NO. 220 |
| CkIIbeta | HFA20230 | −2.7 | −2.6 | Signal transduction | IPR000704 | GO: 0004702; receptor signaling protein serine/threonine kinase activity | SEQ ID NO. 221 |
| comm3 | HFA09995 | −2.2 | −2.2 | Unknown | noIPR | na; na | SEQ ID NO. 222 |
| CtBP | HFA16617 | −2.9 | −2.8 | Transcription regulators | IPR006139 | GO: 0003714; transcription corepressor activity | SEQ ID NO. 223 |
| dome | HFA19583 | −6.2 | −4.9 | Signal transduction | IPR000194 | GO: 0004907; interleukin receptor activity | SEQ ID NO. 224 |
| eIF-4B | HFA20983 | −3.2 | −3.0 | RNA processing and Translation | IPR000504 | GO: 0003723; RNA binding | SEQ ID NO. 225 |
| HDC01676 | HFA01091 | −2.3 | −2.6 | Unknown | IPR006202 | na; na | SEQ ID NO. 226 |
| HDC11198 | HFA11427 | −2.3 | −2.2 | Unknown | noIPR | na; na | SEQ ID NO. 227 |
| hop | HFA20340 | −5.7 | −4.1 | Signal transduction | IPR001245 | GO: 0004718; Janus kinase activity | SEQ ID NO. 228 |
| Ipk2 | HFA00357 | −2.6 | −4.0 | Signal transduction | IPR005522 | GO: 0050516; inositol-polyphosphate multikinase activity | SEQ ID NO. 229 |
| jbug | HFA04167 | −2.7 | −3.2 | Cytoskeleton and Transport | IPR001298 | GO: 0005200; structural constituent of cytoskeleton | SEQ ID NO. 230 |
| kn | HFA07637 | −2.4 | −2.4 | Transcription regulators | IPR003523 | GO: 0030528; transcription regulator activity | SEQ ID NO. 231 |
| l(1)G0084 | HFA19450 | −2.1 | −2.1 | Transcription regulators | IPR001965 | GO: 0003677; DNA binding | SEQ ID NO. 232 |
| larp | HFA16984 | −2.5 | −2.4 | Unknown | IPR006630 | na; na | SEQ ID NO. 233 |
| mask | HFA15370 | −2.3 | −2.7 | Signal transduction | IPR002110 | GO: 0005102; receptor binding | SEQ ID NO. 234 |
| mst | HFA20582 | −2.2 | −2.6 | Unknown | noIPR | na; na | SEQ ID NO. 235 |
| nonA | HFA20357 | −3.0 | −3.3 | RNA processing and Translation | IPR000504 | GO: 0030528; transcription regulator activity | SEQ ID NO. 236 |
| Obp93a | HFA15220 | −2.4 | −2.9 | Cytoskeleton and Transport | IPR006170 | GO: 0005549; odorant binding | SEQ ID NO. 237 |
| Rrp1 | HFA00784 | −4.3 | −4.3 | Others | IPR000097 | GO: 0004520; endodeoxyribonuclease activity | SEQ ID NO. 238 |
| sol | HFA20587 | −2.5 | −3.0 | Others | IPR001876 | GO: 0005516; calmodulin binding | SEQ ID NO. 239 |
| Stat92E | HFA16870 | −5.0 | −5.2 | Signal transduction | IPR001217 | GO: 0004871; signal transducer activity | SEQ ID NO. 240 |
| Taf2 | HFA11298 | −2.7 | −2.9 | Transcription regulators | IPR002052 | GO: 0016251; general RNA polymerase II transcription factor activity | SEQ ID NO. 241 |

Negative regulators

| | | | | | | | |
|---|---|---|---|---|---|---|---|
| bon | HFA16914 | 5.6 | 4.8 | Protein modifying enzymes/Metabolism | IPR001841 | GO: 0004842; ubiquitin-protein ligase activity | SEQ ID NO. 242 |
| Caf1 | HFA16596 | 3.0 | 2.6 | Protein modifying enzymes/Metabolism | IPR001680 | GO: 0035035; histone acetyltransferase binding | SEQ ID NO. 243 |
| CG10077 | HFA09691 | 2.8 | 4.0 | RNA processing and Translation | IPR001410 | GO: 0003724; RNA helicase activity | SEQ ID NO. 244 |
| CG11400 | HFA06070 | 2.6 | 2.2 | Unknown | noIPR | na; na | SEQ ID NO. 245 |
| CG11501 | HFA14317 | 3.7 | 3.1 | Unknown | noIPR | na; na | SEQ ID NO. 246 |
| CG13499 | HFA04144 | 2.5 | 3.1 | Unknown | noIPR | na; na | SEQ ID NO. 247 |
| CG14247 | HFA14742 | 3.2 | 3.4 | Unknown | IPR002557 | na; na | SEQ ID NO. 248 |
| CG15706 | HFA06577 | 2.2 | 2.1 | Unknown | IPR011701 | na; na | SEQ ID NO. 249 |
| CG16975 | HFA02552 | 2.7 | 2.7 | Transcription regulators | IPR001660 | GO: 0030528; transcription regulator activity | SEQ ID NO. 250 |
| CG17492 | HFA02623 | 2.5 | 2.1 | Protein modifying enzymes/Metabolism | IPR001841 | GO: 0004842; ubiquitin-protein ligase activity | SEQ ID NO. 251 |
| CG18112 | HFA15304 | 2.1 | 2.1 | Unknown | IPR001829 | na; na | SEQ ID NO. 252 |
| CG30122 | HFA06935 | 3.3 | 2.8 | Transcription regulators | IPR003034 | GO: 0003677; DNA binding | SEQ ID NO. 253 |
| CG4907 | HFA15673 | 3.3 | 3.5 | Unknown | IPR007070 | na; na | SEQ ID NO. 254 |
| dre4 | HFA08714 | 2.6 | 2.5 | Transcription regulators | IPR000994 | GO: 0003712; transcription cofactor activity | SEQ ID NO. 255 |
| enok | HFA04096 | 3.0 | 3.0 | Transcription regulators | IPR001965 | GO: 0030528; transcription regulator activity | SEQ ID NO. 256 |
| lig | HFA07247 | 2.2 | 2.1 | Unknown | IPR009060 | na; na | SEQ ID NO. 257 |
| Nup154 | HFA03384 | 2.9 | 2.9 | Cytoskeleton and Transport | IPR011045 | GO: 0005487; nucleocytoplasmic transporter activity | SEQ ID NO. 258 |
| par-1 | HFA07660 | 4.4 | 4.2 | Signal transduction | IPR000719 | GO: 0004674; protein serine/threonine kinase activity | SEQ ID NO. 259 |
| Pp1alpha-96A | HFA16795 | 3.0 | 3.8 | Signal transduction | IPR006186 | GO: 0004722; protein serine/threonine phosphatase activity | SEQ ID NO. 260 |
| PP2A-B′ | HFA16344 | 2.6 | 2.5 | Signal transduction | IPR002554 | GO: 0008601; protein phosphatase type 2A regulator activity | SEQ ID NO. 261 |
| Ptp61F | HFA08683 | 5.9 | 8.1 | Signal transduction | IPR000863 | GO: 0004725; protein tyrosine phosphatase activity | SEQ ID NO. 262 |
| Rab5 | HFA00777 | 2.1 | 2.1 | Signal transduction | IPR001806 | GO: 0005525; GTP binding | SEQ ID NO. 263 |
| Socs36E | HFA02455 | 3.2 | 2.3 | Signal transduction | IPR000980 | GO: 0007259; JAK-STAT cascade | SEQ ID NO. 264 |
| TSG101 | HFA11098 | 3.1 | 3.4 | Protein modifying enzymes/Metabolism | IPR001440 | GO: 0004842; ubiquitin-protein ligase activity | SEQ ID NO. 265 |

InterPro Evidence was obtained from: Mulder et al. (2005). InterPro, progress and status in 2005.
GO Evidence was obtained from: R. A. Drysdale, M. A. Crosby and The FlyBase Consortium (2005). FlyBase: genes and gene models. Nucleic Acids Research 33: D390-D395. http://flybase.org/
All 384-well screening plates contained dsRNAs against known JAK/STAT pathway components.
Controls for the 57 screening plates were stat92E RNAI (identified 55 times), hop RNAi (identified 37 times), dome RNAi (identified 55 times) and socs36E RNAi (identified 45 times)
An Interactive table with links to the Interpro records is available at http://www.dkfz.de/signaling/jak-pathway/

TABLE 2
Functional groups classified by InterPro prediction and GO.

| Functional Group^{†} | N* |
|---|---|
| Signalling factors | 17 |
| Transcription factors | 14 |
| Protein modification and Metabolism | 12 |
| Cytoskeleton and Transport | 7 |
| All others | 9 |
| Predicted proteins classified as part of a | 59 |
| Predicted proteins without classification | 31 |

Queries were performed with InterPro 8.0
^{†}InterPro and GO results classified into one of functionally related groups. See Table 1 for complete list of genes, specific IPR domains and GO assigned within each group.
*Number of proteins identified with InterPro domains and/or GO found in 90 translated gene sequences.

SUPPLEMENTARY TABLE 3
Genetic interactions with hop^{Tuml (1)}

| | | Insert/ | Tumours (%) | | | | |
|---|---|---|---|---|---|---|---|
| Exp | Genotype | Allele | None | Small | Large | n | z-score |
| I | y, w, hop^{Tuml}/+; +/+ | OreR | 31.0 | 50.6 | 18.4 | 358 | −0.4⁽*⁾ |
| II | y, w, hop^{Tuml}/+; +/+ | OreR | 31.0 | 43.8 | 25.2 | 445 | −0.4⁽*⁾ |
| II | y, w, hop^{Tuml}/+; +/+ | w1118 | 23.9 | 31.2 | 44.9 | 356 | 0.6⁽*⁾ |
| II | y, w, hop^{Tuml}/+; STAT92E/+ | 397 | 67.5 | 21.5 | 11.0 | 228 | −5.3⁽²⁾ |
| I | y, w, hop^{Tuml}/+; STAT92E/+ | 06346 | 68.6 | 26.1 | 5.3 | 283 | −5.4⁽³⁾ |
| II | y, w, hop^{Tuml}/+; STAT92E/+ | 06346 | 64.2 | 26.6 | 9.2 | 282 | −4.9⁽³⁾ |
| II | y, w, hop^{Tuml}/+; dBRWD3/+ | 05842 | 56.6 | 24.4 | 19.0 | 221 | −3.8 |
| I | y, w, hop^{Tuml}/+; cg-Gal4/UAS-EGFP | 5a.2 | 19.9 | 35.1 | 45.0 | 151 | 1.2⁽*⁾ |
| II | y, w, hop^{Tuml}/+; cg-Gal4/UAS-EGFP | 6a.3 | 41.0 | 33.3 | 25.7 | 451 | −1.7⁽*⁾ |
| II | y, w, hop^{Tuml}/+; cg-Gal4/UAS-lacZ | BG4-1-2 | 25.8 | 26.4 | 47.8 | 341 | 0.4⁽*⁾ |
| II | y, w, hop^{Tuml}/+; cg-Gal4/UAS-ptp61Fa | 1b.2 | 46.5 | 27.7 | 25.7 | 101 | −2.5 |
| I | y, w, hop^{Tuml}/+; cg-Gal4/UAS-ptp61Fa | 1b.2 | 46.5 | 29.1 | 24.3 | 230 | −2.5 |
| I | y, w, hop^{Tuml}/+; cg-Gal4/UAS-ptp61Fa | 1a.3 | 22.6 | 28.8 | 48.6 | 177 | 0.8 |
| II | y, w, hop^{Tuml}/+; cg-Gal4/UAS-ptp61Fa | 1a.3 | 19.6 | 24.4 | 56.0 | 168 | 1.2 |
| II | y, w, hop^{Tuml}/+; cg-Gal4/UAS-ptp61Fa | 3a.3 | 35.8 | 28.5 | 35.8 | 165 | −1.0 |
| I | y, w, hop^{Tuml}/+; cg-Gal4/UAS-ptp61Fa | 7a.3 | 16.4 | 36.1 | 47.5 | 61 | 1.6 |
| II | y, w, hop^{Tuml}/+; cg-Gal4/UAS-ptp61Fc | 1a.1 | 68.2 | 21.4 | 10.4 | 280 | −5.4 |
| II | y, w, hop^{Tuml}/+; cg-Gal4/UAS-ptp61Fc | 2a.4 | 56.1 | 30.6 | 13.3 | 255 | −3.8 |
| I | y, w, hop^{Tuml}/+; cg-Gal4/UAS-ptp61Fc | 2a.4 | 52.3 | 40.7 | 7.0 | 344 | −3.2 |
| I | y, w, hop^{Tuml}/+; cg-Gal4/UAS-ptp61Fc | 2b.3 | 59.4 | 33.8 | 6.8 | 234 | −4.2 |
| II | y, w, hop^{Tuml}/+; cg-Gal4/UAS-ptp61Fc | 2b.3 | 63.3 | 29.3 | 7.3 | 300 | −4.7 |
| II | y, w, hop^{Tuml}/+; cg-Gal4/UAS-dPIAS-GFP | 26b.3 | 67.0 | 27.4 | 5.7 | 106 | −5.2⁽⁴⁾ |
| I | y, w, hop^{Tuml}/+; cg-Gal4/UAS-dPIAS-GFP | 26b.3 | 63.1 | 33.6 | 3.3 | 122 | −4.7⁽⁴⁾ |

Values shown represent percentage of 0-24 hr old female flies containing no, small or large tumours visible in abdomen or thorax. Table shows results from two independent experiments (first column) undertaken under identical conditions.
⁽*⁾‘wild type’ results used to calculate z-scores
References:
⁽¹⁾Hanratty, W. P. & Dearolf, C. R. The Drosophila Tumorous-lethal hematopoietic oncogene is a dominant mutation in the hopscotch locus. Mol Gen Genet 238, 33-7 (1993).
⁽²⁾Silver, D. L. & Montell, D. J. Paracrine signaling through the JAK/STAT pathway activates invasive behavior of ovarian epithelial cells in Drosophila. Cell 107, 831-841 (2001).
⁽³⁾Hou, X. S., Melnick, M. B. & Perrimon, N. Marelle acts downstream of the Drosophila HOP/JAK kinase and encodes a protein similar to the mammalian STATs. Cell 84, 411-9 (1996).
⁽⁴⁾Betz, A., Lampen, N, Martinek, S., Young, M. W. & Darnell, J. E. Jr. A Drosophila PIAS homologue negatively regulates stat92E. Proc Natl Acad Sci USA 98, 9563-8 (2001).

TABLE 4
Sequence and cytological information

| dsRNA ID | Amplicon primer 1A | Ampilcon primer 2 |
|---|---|---|
| HFA00627 | TGC CTG TTT TCT GGA AAT ATG | CTC GCT GGG TTT CAT GGT |
| HFA11324 | TCG AAC TCA CGT TCG AGT | ATC ATC TTC GGG ATG GAT AAC |
| HFA04919 | GAG ATA CCC CGT GAT GAC A | CTT GGG AAT ACG CAC AAA GA |
| HFA16914 | AGG TGC TGG TGG AAA AGA A | ACC CGT CAC CCG GAA AG |
| HFA16596 | TAT TTG CTG TCA GCC TCT G | TGG TCC GTC CTC AGC ATC |
| HFA14173 | CGC CCT GAT CTT TGT GGG | GGA CGA GTA CAT CGC AAT G |
| HFA09691 | GCA CCA CCT CGT TGA AGA | GGG CAG CCA CAT CGG T |
| HFA02102 | GAA CTT CAT TTG GAA GCG TTT | CTT GCG CCG GAA CCA G |
| HFA09807 | GCC GCC GGT ACC GTC | AAG TAG GTG GGC GAT TCC |
| HFA11648 | CCG TGG CCA CAG GAA CA | CAG TCC TGT TCA TGT GGA AAT |
| HFA06070 | TTG TCT GGC TGT GTC TGT C | GAC AAT CCT TGG CCC AAT AAC |
| HFA14317 | ATG GCA TCC CCA GTA GTC A | GTG ACT TTG ATG ATC TGG ATT C |
| HFA19417 | GCC GAC GAA CAG CCA AA | TCG CAC ACC TCG GGA C |
| HFA14478 | TAA CGG TGA CGG AAC CCA | CCG AAT CCT CGA TGG GTT |
| HFA20970 | GCC AAA ATC AAG CGA ATC AG | CTT AAT TGC CTG CAC CTC C |
| HFA19459 | ATC GGC TGC GTG AGA AC | TTC GTT GGC CAA ACT TTA CA |
| HFA01920 | GAT TGG ACG CTT CGC TTT GA | GTT GAA ACA TTG CTG GGT GA |
| HFA10017 | TGG CTG CCA TGC AGA AG | CCA ATT TCG GCA CGG TAG |
| HFA04144 | AGT GGC AGC GGA GGT G | GCC CTC GCA GTG GGT T |
| HEA14742 | AAA ATA AAT GGA GTA ACT TCC CC | TAC GCC TCG CAC TCC A |
| HFA17927 | CGC AAT GTG GAG GTG AAG | ATC GAA ATA CGA GCC GAT C |
| HFA17993 | TTC GAG GGC CCA CAA TGT | TGG CAA GTC GCA ACT TTA C |
| HFA00432 | CAA AGG CAC CTG GTT TGT G | CAG TAG CGC AGA CGT TG |
| HFA00449 | GGT ATT ACT CTG TTC CGA TTG | CTT CCA GGT TTT TGT GTA TGT C |
| HEA15093 | GGC AAA GAT CCC AAG CAG | GTT GAA GGT GCA GCA GAA G |
| HFA06577 | CAG CCA TCG ATT GGA ACA G | CTC CAA GTG CCA GAA CAT AAA |
| HFA18090 | GGC CAC AAG CAT GGT CG | CCT TGC CCT TGC ACT TCT |
| HFA18561 | TCG CCC ATG GTG CTA GA | CGA TCC ACG GTG ATT ACA G |
| HFA02552 | CAG ACT CCT ACC TCG TTT TG | AAC ATG CGC TCC AGA TAG T |
| HFA10258 | GCC AAG AGA CGG AGA AGA | TAC GGA TGC TGG TTG ATG T |
| HFA02623 | CCC AGG GCC ATT TGG ATT T | TCC TTT AAG CGC TGC ATG |
| HFA15304 | GGG CAT GCC GTC ATT ACA | CGG CGA TAT TTG CTG GTC |
| HFA21006 | GTG GCG CAC CGG AAA G | GAT GAA CTT CAT TGT TGT TGA AA |
| HFA06272 | TGA CGA AGC ATA TAC AAG GAT A | TGG GTT TTT CTG GTG AAA CAA |
| HFA06935 | GTT TGC ATC GGC CAA ACC | GTG TCA GAG AAA TTC ACT AAG TA |
| HFA00563 | AAT ACG TTT CGG TCA CGA TT | GTA TCT GTA CTT GGT AGA GTA GT |
| HFA15507 | CCC CGA GCT GAA TCC CA | CTT CAT GCG GTT GAT GAC TA |
| HFA15369 | CGT AAG TGC TAG TTC CTC TG | TGC CGA GCG TCC CTT T |
| HFA16032 | CCC ACG GAG CTG TTC TTT | AAA CGA CTA CCC AGG ACA TT |
| HFA15235 | AGG CAT CTG CAG ATT CTC T | GAG GAA TGG GAA TGG ATG AAG |
| HFA00415 | GTC ATG GGT CCC GGG ATG | TCG CTT GTC ACG ATT CTT T |
| HFA09966 | CCG CCA CAA TGA TAA CCA AC | CGC GTG CGT GAA GAG T |
| HFA19906 | ATC TGT TGA ACG CCG AGG | GGT ATC GGT GAA GTT CTT CTC |
| HFA15470 | TTG TCG CGA CCT TCC CA | ACT TCT TGG AGC AGA TCT TG |
| HFA10378 | CGG ACA CCG GCT ATG TG | ATG TTC TTG GCC GAG TCA A |
| HFA10395 | TAC TCA AGG ATC GCG ATA TC | GGC TGG GTG TGG GAG TG |
| HFA20930 | GCA GGA CGT TCG GAA TAT C | TCC CAT TAC AGA CTT TTG ATT G |
| HEA19892 | GGC GGC ACA TGT GCA TG | GCC GCT GCC CAT ATA CTT |
| HFA10420 | TGT GGC TGT CGC TTA TCT T | AAA AAT ATA CAG CCG TTT CCT T |
| HEA19909 | ACC CAG CTA AAT CCT ACA ATG | ACT CCA GAT GCT GGG TCA |
| HFA04488 | TTG ACG GAT TGC CAC ATC T | GCC TCC GCG TCC AAG T |
| HFA15673 | TGG GCT CGG CAG AGA TA | CAA GTA GAG GAG CCC GAT |
| HFA16036 | TCT TTG TCA TCA AAT CGT ACT C | CAT CGG GCC CAT GCA TT |
| HFA10635 | TTG AAC ATC GTG GCT TCT TT | CCT CGC AAA CTC GAT GC |
| HFA16145 | CAA CAA CAT GCT GGG CTT C | CGA AGT TCG AGC CGA CA |
| HFA20054 | GAG CGG GCG ATC ATC TT | CTC GGC GGC GAT CAC |
| HFA09675 | GAT GAG AAG GAC GAG AAG AG | CTT GAT GCG GCA ATG GAC |
| HFA20148 | ATA GGT TCA ACA CGA TCC CC | GAA GGC TGG TGT TAG TTT TG |
| HFA11946 | ACT TGC GTG GAG GAA CTA A | ATG CGT AGA GTT CTT CGG T |
| HFA20230 | AGC TCG AGG ACA ATC CAC | GGC TGA CTT TCA CAG TAG AC |
| HFA09995 | CGT ACG ATG ATG CAC TGG | GAA CGG GCA GAA TGG TTG |
| HFA16617 | GGC AGT GGG AGC TCT GA | CTC GGG TCC GGT GAA CT |
| HFA19583 | CGT CTG CGC AGT GAT CC | TGG GCT CCG ATG GAT AGA |
| HFA08714 | AGC GAC GAG GAA GAT GTG | TGA CAA ATG TGG CCT CTG G |
| HFA20983 | TTG GAA AAT CGA GAG GAT TTA A | CAC ATT TTT CGA ATT CAA TTG TC |
| HFA04096 | CGT CTA ATG AGG CAA AGA AAC | CCG TTT TTG CCA CTT TAA CC |
| HFA01091 | TCG TGA TGG TGT TGG TGA C | TCC ACT GAA AGT GCT TTG GT |
| HFA11427 | GGG CGA ATG CAC GGA AT | TGG CAT ACC TCG AAT AAC TG |
| HFA20340 | TAA TCG A CG ATC AGG A AC AG | GTG TGG CCT CGG AGG TG |
| HFA00357 | CGT CCC CCG GTT TTA CG | ATC AGC CAG TCT TGA ATA GTC |
| HFA04167 | ATA AAA GGC GCC AAG GTG A | TCA CCT GCA TTC CCG TTT C |
| HFA07637 | GAC GGG CTT CAA TTC CTA TG | GCG ACG AGG AGA GTG TG |
| HFA19450 | TGC TGC GCA AGC GAC | CAT TTG CGT GGA AGA TGA CA |
| HFA16984 | CAC AAA GCC GCT GAA CAG | TTC GTG GTT ACA CAC ACA GT |
| HFA07247 | CCG CGC GAA CGA CTT | TGA TCG CTT ATC ATC GTA TAT TA |
| HFA15370 | ACT AGT AGC AGT CAG TCC TC | GCG CCA GCG TTG CTA T |
| HFA20582 | ACA GCA TTC GGG TGG TAA A | GCC ATC CGA AGT TGA TCG |
| HFA20357 | AAC CAG AAC CAG AAT CAA AAT G | GTT TCC AGC GCG ATT ATT G |
| HFA03384 | GGC TGG ATG GAG TTG TTT G | GGA CTT ATG GGC TGA TTG AAC |
| HFA15220 | AGC GGG TGC AGG AGT TC | TTC TTA TTA CTG GCC ACA TCA T |
| HFA07660 | CAC GTT CTG CGG TAG CC | GCT TGG GAT CGG CTA AAT C |
| HFA16795 | TTG TGG GTA AAT TTT TAC AGA AG | CGA ATT CCC CGC AGT AGT |
| HFA16344 | CGG ATC CGG AGC ACC C | GCG ATG GAG CTG CTG G |
| HFA08683 | CTT GAC GCT GAA GAA CCC | CCT GGA ATT GGA TCG ATG C |
| HFA00777 | GGC AAC CAC TCC ACG CA | TCC TGG CCA GCC GTG T |
| HFA00784 | AGA GCC GCC GAA ACA AC | GGC TTG GTT TCA GTA GAG G |
| HFA02455 | CAG CAG TAA AGC ACT TTC AA | CCG ATT CCG GCA TGG C |
| HFA20587 | GAG TAC AAG CAT GTG TAC AAG | GTT CCT GGT GGA GGT AGT G |
| HFA16870 | CTT GCC CAA AAC TAC AGT TAC | CGA CTG TGG GTG GAT TGT T |
| HEA11298 | AAG GAA AGC GCA TTT CGT | AAA TCC ATA TCC ACT TCC TCA C |
| HFA11098 | ATC CCT CAA ATC CCA GTT CC | AAA GTG GCG CTG TGG TG |

| | No of | Target | Cyto- | |
|---|---|---|---|---|
| | efficient | gene | logical | |
| dsRNA ID | siRNAsB | (Symbol) | location | SEQ ID NOs. |
| HFA00627 | 51/496 | Art2 | 24E1 | SEQ ID NOs. |
| | | | | 266/267 |
| HFA11324 | 61/489 | asf1 | 76B9 | SEQ ID NOs. |
| | | | | 268/269 |
| HFA04919 | 87/487 | bin3 | 42A13--14 | SEQ ID NOs. |
| | | | | 270/271 |
| HFA16914 | 60/496 | bon | 92F2--3 | SEQ ID NOs. |
| | | | | 272/273 |
| HFA16596 | 81/496 | Caf1 | 88E3 | SEQ ID NOs. |
| | | | | 274/275 |
| HFA14173 | 139/494 | CG10007 | 87A4 | SEQ ID NOs. |
| | | | | 276/277 |
| HFA09691 | 72/484 | CG10077 | 65D3--4 | SEQ ID NOs. |
| | | | | 278/279 |
| HFA02102 | 103/497 | CG10730 | 38B2 | SEQ ID NOs. |
| | | | | 280/281 |
| HFA09807 | 63/495 | CG10960 | 69E5--6 | SEQ ID NOs. |
| | | | | 282/283 |
| HFA11648 | 51/242 | CG11307 | 78E1 | SEQ ID NOs. |
| | | | | 284/285 |
| HFA06070 | 85/459 | CG11400 | 54A1 | SEQ ID NOs. |
| | | | | 286/287 |
| HFA14317 | 28/312 | CG11501 | 99B1 | SEQ ID NOs. |
| | | | | 288/289 |
| HFA19417 | 64/486 | CG11696 | 10C7 | SEQ ID NOs. |
| | | | | 290/291 |
| HFA14478 | 78/498 | CG12213 | 87A3 | SEQ ID NOs. |
| | | | | 292/293 |
| HFA20970 | 50/114 | CG12460* | hetero- | SEQ ID NOs. |
| | | | chromatin | 294/295 |
| HFA19459 | 19/181 | CG12479 | 12E2 | SEQ ID NOs. |
| | | | | 296/297 |
| HFA01920 | 112/494 | CG13243 | 35D4--5 | SEQ ID NOs. |
| | | | | 298/299 |
| HFA10017 | 73/391 | CG13473 | 70F3 | SEQ ID NOs. |
| | | | | 300/301 |
| HFA04144 | 19/256 | CG13499 | 58B1 | SEQ ID NOs. |
| | | | | 302/303 |
| HEA14742 | 34/497 | CG14247 | 97D1 | SEQ ID NOs. |
| | | | | 304/305 |
| HFA17927 | 52/490 | CG14434 | 6D7 | SEQ ID NOs. |
| | | | | 306/307 |
| HFA17993 | 122/475 | CG15306 | 9B7 | SEQ ID NOs. |
| | | | | 308/309 |
| HFA00432 | 19/143 | CG15418 | 24A2 | SEQ ID NOs. |
| | | | | 310/311 |
| HFA00449 | 30/217 | CG15434 | 24F3 | SEQ ID NOs. |
| | | | | 312/313 |
| HEA15093 | 58/283 | CG15555 | 100B9 | SEQ ID NOs. |
| | | | | 314/315 |
| HFA06577 | 77/477 | CG15706 | 52F11 | SEQ ID NOs. |
| | | | | 316/317 |
| HFA18090 | 51/500 | CG15784 | 4F10 | SEQ ID NOs. |
| | | | | 318/319 |
| HFA18561 | 72/477 | CG16903 | 2C10 | SEQ ID NOs. |
| | | | | 320/321 |
| HFA02552 | 54/495 | CG16975 | 34A7--8 | SEQ ID NOs. |
| | | | | 322/323 |
| HFA10258 | 3/155 | CG17179* | U | SEQ ID NOs. |
| | | | | 324/325 |
| HFA02623 | 71/486 | CG17492 | 37B10--11 | SEQ ID NOs. |
| | | | | 326/327 |
| HFA15304 | 78/475 | CG18112 | 99C2 | SEQ ID NOs. |
| | | | | 328/329 |
| HFA21006 | 50/114 | CG18160* | U | SEQ ID NOs. |
| | | | | 330/331 |
| HFA06272 | 111/489 | CG30069 | 50E2--3 | SEQ ID NOs. |
| | | | | 332/333 |
| HFA06935 | 62/463 | CG30122 | 55E3 | SEQ ID NOs. |
| | | | | 334/335 |
| HFA00563 | 69/326 | CG3058 | 24F1 | SEQ ID NOs. |
| | | | | 336/337 |
| HFA15507 | 8/197 | CG31005 | 100B8 | SEQ ID NOs. |
| | | | | 338/339 |
| HFA15369 | 34/488 | CG31132 | 95F12--13 | SEQ ID NOs. |
| | | | | 340/341 |
| A16032 | 63/495 | CG31132 | 95F12--13 | SEQ ID NOs. |
| | | | | 342/343 |
| HFA15235 | 112/488 | CG31358 | 87A5 | SEQ ID NOs. |
| | | | | 344/345 |
| HFA00415 | 28/159 | CG31694 | 23B7--8 | SEQ ID NOs. |
| | | | | 346/347 |
| HFA09966 | 68/477 | CG32406 | 65A2--3 | SEQ iD NOs. |
| | | | | 348/349 |
| HFA19906 | 39/495 | CG32573 | 14F5 | SEQ ID NOs. |
| | | | | 350/351 |
| HFA15470 | 66/500 | CG3281 | 87A3 | SEQ ID NOs. |
| | | | | 352/353 |
| HFA10378 | 70/482 | CG3819 | 75E6 | SEQ ID NOs. |
| | | | | 354/355 |
| HFA10395 | 53/484 | CG4022 | 6B84--5 | SEQ ID NOs. |
| | | | | 356/357 |
| HFA20930 | 199/540 | CG40351 | U | SEQ ID NOs. |
| | | | | 358/359 |
| HEA19892 | 97/480 | CG4349 | 11D11 | SEQ ID NOs. |
| | | | | 360/361 |
| HFA10420 | 56/481 | CG4446 | 67B2 | SEQ ID NOs. |
| | | | | 362/363 |
| HEA19909 | 55/496 | CG4653 | 15A3 | SEQ ID NOs. |
| | | | | 364/365 |
| HFA04488 | 75/482 | CG4781 | 60D10 | SEQ ID NOs. |
| | | | | 366/367 |
| HFA15673 | 105/492 | CG4907 | 94C2 | SEQ ID NOs. |
| | | | | 368/369 |
| HFA16036 | 102/487 | CG6422 | 96B17 | SEQ ID NOs. |
| | | | | 370/371 |
| HFA10635 | 39/148 | CG6434 | 77B4 | SEQ ID NOs. |
| | | | | 372/373 |
| HFA16145 | 118/468 | CG6946 | 86F8--9 | SEQ ID NOs. |
| | | | | 374/375 |
| HFA20054 | 33/452 | CG7635 | 18A6 | SEQ ID NOs. |
| | | | | 376/377 |
| HFA09675 | 56/481 | CG8108 | 67C11--D1 | SEQ ID NOs. |
| | | | | 378/379 |
| HFA20148 | 93/492 | CG9086 | 15C5--6 | SEQ ID NOs. |
| | | | | 380/381 |
| HFA11946 | 144/490 | Ckllalpha | 80D1 | SEQ ID NOs. |
| | | | | 382/383 |
| HFA20230 | 27/152 | Ckllbeta | 10E3 | SEQ ID NOs. |
| | | | | 384/385 |
| HFA09995 | 37/499 | comm3 | 71E3--4 | SEQ ID NOs. |
| | | | | 386/387 |
| HFA16617 | 30/254 | CtBP | 87D8--9 | SEQ ID NOs. |
| | | | | 388/389 |
| HFA19583 | 39/480 | dome | 18D13--E1 | SEQ ID NOs. |
| | | | | 390/391 |
| HFA08714 | 65/476 | dre4 | 62B7 | SEQ ID NOs. |
| | | | | 392/393 |
| HFA20983 | 159/488 | elF-4B | U | SEQ ID NOs. |
| | | | | 394/395 |
| HFA04096 | 84/487 | enok | 60B10 | SEQ ID NOs. |
| | | | | 396/397 |
| HFA01091 | 48/220 | HDCC1676 | 30D1 | SEQ ID NOs. |
| | | | | 398/399 |
| HFA11427 | 105/477 | HDC11198 | 77D4 | SEQ ID NOs. |
| | | | | 400/401 |
| HFA20340 | 52/493 | hop | 10B5--6 | SEQ ID NOs. |
| | | | | 402/403 |
| HFA00357 | 75/488 | lpk2 | 21E2 | SEQ ID NOs. |
| | | | | 404/405 |
| HFA04167 | 18/202 | jbug | 59A3 | SEQ ID NOs. |
| | | | | 406/407 |
| HFA07637 | 10/228 | kn | 51C2--3 | SEQ ID NOs. |
| | | | | 408/409 |
| HFA19450 | 38/496 | l(1)G0084 | 18D8--11 | SEQ ID NOs. |
| | | | | 410/411 |
| HFA16984 | 88/496 | larp | 98C3--4 | SEQ ID NOs. |
| | | | | 412/413 |
| HFA07247 | 35/377 | lig | 44A4 | SEQ ID NOs. |
| | | | | 414/415 |
| HFA15370 | 30/486 | mask | 95F3--5 | SEQ ID NOs. |
| | | | | 416/417 |
| HFA20582 | 58/473 | mst | 20A1 | SEQ ID NOs. |
| | | | | 418/419 |
| HFA20357 | 27/118 | nonA | 14B18--C1 | SEQ ID NOs. |
| | | | | 420/421 |
| HFA03384 | 87/500 | Nup154 | 32C5 | SEQ ID NOs. |
| | | | | 422/423 |
| HFA15220 | 36/167 | Obp93a | 93C1 | SEQ ID NOs. |
| | | | | 424/425 |
| HFA07660 | 60/324 | par-1 | 56D9--11 | SEQ ID NOs. |
| | | | | 426/427 |
| HFA16795 | 12/118 | Pp1alpha-96A | 96A5 | SEQ ID NOs. |
| | | | | 428/429 |
| HFA16344 | 32/469 | PP2A-B′ | 90E4--5 | SEQ ID NOs. |
| | | | | 430/431 |
| HFA08683 | 72/495 | Ptp61F | 61F7--62A1 | SEQ ID NOs. |
| | | | | 432/433 |
| HFA00777 | 28/244 | Rab5 | 22E1 | SEQ ID NOS. |
| | | | | 434/435 |
| HFA00784 | 98/487 | Rrp1 | 23C3--4 | SEQ ID NOs. |
| | | | | 436/437 |
| HFA02455 | 38/490 | Socs36E | 36E6 | SEQ ID NOs. |
| | | | | 438/439 |
| HFA20587 | 31/359 | sol | 19F5 | SEQ ID NOs. |
| | | | | 440/441 |
| HFA16870 | 64/479 | Stat92E | 92F1 | SEQ ID NOs. |
| | | | | 442/443 |
| HEA11298 | 114/481 | Taf2 | 67D1 | SEQ ID NOs. |
| | | | | 444/445 |
| HFA11098 | 53/319 | TSG101 | 73D1 | SEQ ID NOs. |
| | | | | 446/447 |

A Complete amplicon informaion can be obtained at http://mai.dkfz.de
B Efficiency calculated based on Reynolds et al., (2004). All siRNAs with score of 6 or higher were counted as efficient.
*Annotation according to Release 2 of the Berkeley Drosophila Genome Project

TABLE 5
Human homologues of Drosophila genes with JAK/STAT phenotypes

| | | | | | RefSeq | |
|---|---|---|---|---|---|---|
| Drosophila | | Identity | | | nucleic | |
| Gene | BLASTP | [%] | Human Gene | RefSeq protein | acid | SEQ ID Nos |
| Art2 | 1.60E−77 | 44.2 | protein arginine N-methyltransferase 4 | NP_062828.2 | NM_019854 | SEQ ID NO. 1/88 |
| asf1 | 3.20E−68 | 61.7 | ASF1 anti-silencing function 1 homolog A | NP_054753.1 | NM_014034 | SEQ ID NO. 2/89 |
| bin3 | 8.80E−49 | 34.3 | hypothetical protein FLJ20257 | NP_062552.2 | NM_019606 | SEQ ID NO. 3/90 |
| bon | 3.60E−45 | 30.5 | tripartite motif-containing 33 protein | NP_056990.2 | NM_015906 | SEQ ID NO. 4/91 |
| Caf1 | 0 | 91.9 | retinoblastoma binding protein 4 | NP_005601.1 | NM_005610 | SEQ ID NO. 5/92 |
| CG10007 | 8.80E−50 | 34.5 | chromosome 2 open reading frame 18 | NP_060347.2 | NM_017877 | SEQ ID NO. 6/93 |
| CG10077 | 7.00E−171 | 67.7 | DEAD (Asp-Glu-Ala-Asp) box polypeptide 5 | NP_004387.1 | NM_004396 | SEQ ID NO. 7/94 |
| CG10960 | 3.40E−78 | 36.9 | solute carrier family 2, (facilitated glucose | NP_055395.2 | NM_014580 | SEQ ID NO. 8/95 |
| | | | transporter) member 8 | | | |
| CG11696 | 5.10E−33 | 29.9 | zinc finger protein 502 | NP_149987.2 | NM_033210 | SEQ ID NO. 9/96 |
| CG12460 | 1.80E−17 | 54.0 | splicing factor proline/glutamine rich (polypyrimidine | NP_005057.1 | NM_005066 | SEQ ID NO. 10/97 |
| | | | tract binding protein associated) | | | |
| CG13473 | 3.90E−17 | 34.9 | thioredoxin 2 precursor | NP_036605.2 | NM_012473 | SEQ ID NO. 11/98 |
| CG15306 | 3.30E−27 | 45.1 | microtubule-associated protein, RP/EB family, member 1 | NP_036457.1 | NM_012325 | SEQ ID NO. 12/99 |
| CG15418 | 1.40E−10 | 41.1 | tissue factor pathway inhibitor 2 | NP_006519.1 | NM_006528 | SEQ ID NO. 13/100 |
| CG15434 | 1.60E−17 | 50.6 | NADH dehydrogenase (ubiquinone) | NP_002479.1 | NM_002488 | SEQ ID NO. 14/101 |
| | | | 1 alpha subcomplex, 2, 8 kDa | | | |
| CG15706 | 6.60E−20 | 20.0 | FLJ20160 protein | NP_060164.2 | NM_017694 | SEQ ID NO. 15/102 |
| CG16903 | 2.00E−100 | 63.1 | cyclin L1 | NP_064703.1 | NM_020307 | SEQ ID NO. 16/103 |
| CG16975 | 4.00E−123 | 49.4 | l(3)mbt-like 2 isoform a | NP_113676.2 | NM_031488 | SEQ ID NO. 17/104 |
| CG17492 | 0 | 48.3 | zinc finger, ZZ type with ankyrin repeat domain 1 | NP_543151.1 | NM_080875 | SEQ ID NO. 18/105 |
| CG18112 | 1.80E−20 | 27.8 | chromosome 14 open reading frame 133 | NP_071350.2 | NM_022067 | SEQ ID NO. 19/106 |
| CG30122 | 7.20E−42 | 40.9 | E1B-55 kDa-associated protein 5 isoform a | NP_008971.2 | NM_007040 | SEQ ID NO. 20/107 |
| CG3058 | 2.60E−80 | 95.8 | thioredoxin-like 4 | XP_499552.1 | XM_499552 | SEQ ID NO. 21/108 |
| CG31005 | 9.00E−100 | 52.5 | trans-prenyltransferase | NP_055132.2 | NM_014317 | SEQ ID NO. 22/109 |
| CG31132 | 0 | 49.9 | bromo domain-containing protein disrupted in leukemia | NP_694984.2 | NM_153252 | SEQ ID NO. 23/110 |
| CG31358 | 2.10E−51 | 44.2 | stomatin-like 3 | NP_660329.1 | NM_145286 | SEQ ID NO. 24/111 |
| CG31694 | 2.00E−66 | 36.3 | interferon-related developmental regulator 2 | NP_006755.3 | NM_006764 | SEQ ID NO. 25/112 |
| CG32406 | 5.50E−15 | 37.8 | C1 domain-containing phosphatase | NP_938072.1 | NM_198316 | SEQ ID NO. 26/113 |
| | | | and tensin-like protein isoform 3 | | | |
| CG3281 | 4.00E−41 | 31.7 | zinc finger protein 91 | NP_003421.1 | NM_003430 | SEQ ID NO. 27/114 |
| CG40351 | 2.20E−94 | 56.6 | PREDICTED: KIAA1076 protein | XP_037523.9 | XM_037523 | SEQ ID NO. 28/115 |
| CG4349 | 4.60E−35 | 45.2 | ferritin, heavy polypeptide 1 | NP_002023.2 | NM_002032 | SEQ ID NO. 29/116 |
| CG4446 | 1.90E−66 | 47.2 | pyridoxal kinase | NP_003672.1 | NM_003681 | SEQ ID NO. 30/117 |
| CG4653 | 8.80E−23 | 30.7 | protease, serine, 2 preproprotein | NP_002761.1 | NM_002770 | SEQ ID NO. 31/118 |
| CG4781 | 2.00E−17 | 33.8 | PREDICTED: similar to KIAA0644 protein | XP_379800.1 | XM_379800 | SEQ ID NO. 32/119 |
| CG4907 | 1.10E−47 | 28.9 | phosphatidylinositol glycan, class N | NP_036459.1 | NM_012327 | SEQ ID NO. 33/120 |
| CG6422 | 1.30E−71 | 53.8 | YTH domain family, member 1 | NP_060268.2 | NM_017798 | SEQ ID NO. 34/121 |
| CG6434 | 6.20E−69 | 71.2 | retinoblastoma binding protein 5 | NP_005048.2 | NM_005057 | SEQ ID NO. 35/122 |
| CG6946 | 4.00E−39 | 46.2 | heterogeneous nuclear ribonucleoprotein F | NP_004957 | NM_004966 | SEQ ID NO. 36/123 |
| CG7635 | 2.90E−76 | 62.1 | stomatin isoform a | NP_004090.4 | NM_004099 | SEQ ID NO. 37/124 |
| CG9086 | 0 | 32.0 | ubiquitin protein ligase E3 component n-recognin 1 | NP_777576.1 | NM_174916 | SEQ ID NO. 38/125 |
| CkIIalpha | 4.00E−163 | 88.7 | casein kinase II alpha 1 subunit isoform a | NP_001886.1 | NM_001895 | SEQ ID NO. 39/126 |
| CkIIbeta | 5.00E−107 | 89.2 | casein kinase 2, beta polypeptide | NP_001311.3 | NM_001320 | SEQ ID NO. 40/127 |
| CtBP | 8.00E−152 | 72.4 | C-terminal binding protein 2 isoform 1 | NP_001320.1 | NM_001329 | SEQ ID NO. 41/128 |
| dome | 7.60E−15 | 28.2 | sidekick 2 | NP_061937.2 | NM_019064 | SEQ ID NO. 42/129 |
| dre4 | 0 | 59.9 | chromatin-specific transcription elongation | NP_009123.1 | NM_007192 | SEQ ID NO. 43/130 |
| | | | factor large subunit | | | |
| eIF-4B | 4.70E−32 | 27.2 | eukaryotic translation initiation factor 4B | NP_001408.1 | NM_001417 | SEQ ID NO. 44/131 |
| enok | 1.80E−94 | 33.4 | MYST histone acetyltransferase (monocytic leukemia) 3 | NP_006757.1 | NM_006766 | SEQ ID NO. 45/132 |
| HDC01676 | 2.80E−15 | 61.0 | cholinergic receptor, nicotinic, alpha polypeptide | NP_000737.1 | NM_000746 | SEQ ID NO. 46/133 |
| | | | 7 precursor | | | |
| hop | 1.60E−59 | 26.7 | Janus kinase 2 | NP_004963.1 | NM_004972 | SEQ ID NO. 47/134 |
| Ipk2 | 1.80E−26 | 33.6 | inositol polyphosphate multikinase | NP_689416.1 | NM_152230 | SEQ ID NO. 48/135 |
| jbug | 5.30E−45 | 27.6 | filamin B, beta (actin binding protein 278) | NP_001448.1 | NM_001457 | SEQ ID NO. 49/136 |
| kn | 0 | 69.7 | early B-cell factor | NP_076870.1 | NM_024007 | SEQ ID NO. 50/137 |
| l(1)G0084 | 7.40E−28 | 31.5 | PHD finger protein 10 isoform a | NP_060758.1 | NM_018288 | SEQ ID NO. 51/138 |
| larp | 2.00E−103 | 48.1 | KIAA0731 protein | NP_056130.2 | NM_015315 | SEQ ID NO. 52/139 |
| lig | 5.50E−25 | 32.8 | ubiquitin associated protein 2 isoform 2 | NP_065918.1 | NM_020867 | SEQ ID NO. 53/140 |
| mask | 0 | 74.0 | multiple ankyrin repeats, single KH-domain | NP_060217.1 | NM_017747 | SEQ ID NO. 54/141 |
| | | | protein isoform 1 | | | |
| mst | 7.00E−52 | 29.7 | misato | NP_060586.2 | NM_018116 | SEQ ID NO. 55/142 |
| nonA | 1.80E−60 | 40.6 | splicing factor proline/glutamine rich | NP_005057.1 | NM_005066 | SEQ ID NO. 56/143 |
| | | | (polypyrimidine tract binding protein associated) | | | |
| Nup154 | 0 | 32.6 | nucleoporin 155 kDa isoform 1 | NP_705618.1 | NM_153485 | SEQ ID NO. 57/144 |
| par-1 | 0 | 54.1 | MAP/microtubule affinity-regulating kinase 3 | NP_002367.4 | NM_002376 | SEQ ID NO. 58/145 |
| Pp1alpha-96A | 8.00E−169 | 88.9 | protein phosphatase 1, catalytic subunit, alpha isoform 1 | NP_002699.1 | NM_002708 | SEQ ID NO. 59/146 |
| PP2A-B′ | 0 | 78.9 | delta isoform of regulatory subunit B56, | NP_006236.1 | NM_006245 | SEQ ID NO. 60/147 |
| | | | protein phosphatase 2A isoform 1 | | | |
| Ptp61F | 5.00E−32 | 37.9 | hypothetical protein LOC9671 | NP_055468 | NM_014653 | SEQ ID NO. 61/148 |
| Rab5 | 4.90E−85 | 75.0 | RAB5A, member RAS oncogene family | NP_004153.2 | NM_004162 | SEQ ID NO. 62/149 |
| Rrp1 | 6.10E−82 | 55.2 | APEX nuclease | NP_542380.1 | NM_080649 | SEQ ID NO. 63/150 |
| Socs36E | 4.80E−65 | 68.0 | suppressor of cytokine signaling 5 | NP_054730.1 | NM_014011 | SEQ ID NO. 64/151 |
| Stat92E | 6.40E−86 | 41.6 | signal transducer and activator of transcription 5B | NP_036580.2 | NM_012448 | SEQ ID NO. 65/152 |
| Taf2 | 0 | 52.5 | TBP-associated factor 2 | NP_003175.1 | NM_003184 | SEQ ID NO. 66/153 |
| TSG101 | 4.30E−98 | 48.7 | tumor susceptibility gene 101 | NP_006283.1 | NM_006292 | SEQ ID NO. 67/154 |

Shown are human homologues of Drosophila genes with a BLASTP E value of 10⁻¹⁰ or less.

TABLE 6
Human disease homologues of Drosophila genes with JAK/STAT phenotypes

| Drosophila | | | |
|---|---|---|---|
| Gene | BLASTP | Human Gene | RefSeq protein |
| bon | 3.60E−45 | tripartite motif- | NP_056990.2 |
| | | containing 33 protein | |
| Caf1 | 9.90E−17 | peroxin 7 | NP_000279 |
| CG10960 | 1.40E−39 | erythrocyte/hepatoma | NP_006507 |
| | | glucose transporter | |
| CG11696 | 2.60E−25 | zinc finger protein 41 | NP_006051 |
| CG17492 | 4.80E−23 | ankyrin, brain | NP_001139 |
| CG31132 | 2.70E−17 | Lissencephaly-1 Gene | NP_000421 |
| CG31132 | 0 | bromo domain-containing protein disrupted in leukemia | NP_694984 |
| CG31358 | 7.40E−38 | Podocin | NP_055440 |
| CG32573 | 7.80E−47 | Protein Kinase C, alpha | NP_002728 |
| CG3281 | 2.00E−33 | zinc finger protein 41 | NP_009061 |
| CG40351 | 2.10E−14 | Androgen Receptor-Associated Coregulator 267 | NP_071900 |
| CG4349 | 4.60E−35 | ferritin, heavy polypeptide 1 | NP_002023.2 |
| CG4349 | 3.50E−35 | fth | NP_002023 |
| CG4653 | 6.90E−22 | Protease, Serine, 1 | NP_002760 |
| CG7635 | 2.90E−76 | stomatin isoform a | NP_004090.4 |
| CG7635 | 5.70E−62 | Podocin | NP_055440 |
| CkIIalpha | 1.20E−23 | serine/threonine protein kinase 9 | NP_003150 |
| CtBP | 4.20E−24 | 3-phosphogylcerate dehydrogenase; 3pgdh | NP_006614 |
| dre4 | 4.60E−46 | Lipase A precursor | NP_000226 |
| HDC01676 | 2.80E−15 | cholinergic receptor, nicotinic, alpha polypeptide 7 precursor | NP_000737.1 |
| hop | 8.40E−52 | Janus kinase 3 | NP_000206 |
| jbug | 5.30E−45 | filamin B, beta (actin binding protein 278) | NP_001448.1 |
| jbug | 5.30E−45 | filamin B, beta (actin binding protein 278) | NP_001448.1 |
| jbug | 5.30E−45 | filamin B, beta (actin binding protein 278) | NP_001448.1 |
| jbug | 6.00E−102 | actin-binding protein 280; abp280 | NP_001447 |
| mask | 5.30E−59 | ankyrin, brain | NP_001139 |
| par-1 | 5.30E−39 | Oncogene Akt2 | NP_001617 |
| Ptp61F | 7.80E−79 | Protein phosphotyrosylphosphatase 1B | NP_002818 |
| Rab5 | 8.90E−23 | ras-associatad protein RAB27A | NP_004571 |
| sol | 2.80E−33 | calcium-activated neutral protease 3 | NP_000061 |
| Stat92E | 6.40E−86 | signal transducer and activator of transcription 5B | NP_036580.2 |
| Stat92E | 6.40E−86 | signal transducer and activator of transcription 5B | NP_036580.2 |
| TSG101 | 4.30E−98 | tumor susceptibility gene 101 | NP_006283.1 |

| | RefSeq | | |
|---|---|---|---|
| Drosophila | Nucleic | | |
| Gene | acid | Disease | SEQ ID Nos |
| bon | NM_015906 | Thyroid carcinoma, papillary(2) | SEQ ID NO. 4/91 |
| Caf1 | NM_000288 | Refsum disease (1) | SEQ ID NO. 68/155 |
| CG10960 | NM_006516 | Glucose transport defect, blood-brain barrier (1) | SEQ ID NO. 69/156 |
| CG11696 | NM_006060 | Mental Retardation, X-linked nonsyndromic (1) | SEQ ID NO. 70/157 |
| CG17492 | NM_001148 | Long QT syndrome 4 (1) | SEQ ID NO. 71/158 |
| CG31132 | NM_000430 | Subcortical laminar heterotopia (1) | SEQ ID NO. 72/159 |
| CG31132 | NM_153252 | Leukemia (3) | SEQ ID NO. 73/160 |
| CG31358 | NM_014625 | Nephrotic syndrome, steroid-resistant (1) | SEQ ID NO. 74/161 |
| CG32573 | NM_002737 | Pituitary Tumor, invasive (1) | SEQ ID NO. 75/162 |
| CG3281 | NM_007130 | Mental Retardation, X-linked nonsyndromic (1) | SEQ ID NO. 76/163 |
| CG40351 | NM_022455 | Sotos Syndrome, sporadic (1) | SEQ ID NO. 77/164 |
| CG4349 | NM_002032 | Iron overload, autosomal dominant (2) | SEQ ID NO. 29/116 |
| CG4349 | NM_002032 | Iron overload, autosomal dominant (1) | SEQ ID NO. 29/116 |
| CG4653 | NM_002769 | Pancreatitis, hereditary (1) | SEQ ID NO. 78/165 |
| CG7635 | NM_004099 | Stomatocytosis I (2) | SEQ ID NO. 37/124 |
| CG7635 | NM_014625 | Nephrotic syndrome, steroid-resistant (1) | SEQ ID NO. 74/161 |
| CkIIalpha | NM_003159 | Rett Syndrome, atypical (1) | SEQ ID NO. 79/166 |
| CtBP | NM_006623 | Phosphoglycerate dehydrogenase deficiency (1) | SEQ ID NO. 80/167 |
| dre4 | NM_000235 | Wolman disease (1) | SEQ ID NO. 81/168 |
| HDC01676 | NM_000746 | Schizophrenia, neurophysiologic defect in (2) | SEQ ID NO. 46/133 |
| hop | NM_000215 | SCID, autosomal recessive, T-negative/B- | SEQ ID NO. 82/169 |
| | | positive type (1) | |
| jbug | NM_001457 | Atelostogenesis, type I (2) | SEQ ID NO. 49/136 |
| jbug | NM_001457 | Larson syndrome (2) | SEQ ID NO. 49/136 |
| jbug | NM_001457 | Spondylocarpotarsal synostosis syndrome (2) | SEQ ID NO. 49/136 |
| jbug | NM_001456 | Frontometaphyseal dysplasia (1) | SEQ ID NO. 83/170 |
| mask | NM_001148 | Long QT syndrome 4 (1) | SEQ ID NO. 71/158 |
| par-1 | NM_001626 | Diabetes mellitus, type II (1) | SEQ ID NO. 84/171 |
| Ptp61F | NM_002827 | Insulin resistance, susceptibility to (1) | SEQ ID NO. 85/172 |
| Rab5 | NM_004580 | Griscelli Syndrome (1) | SEQ ID NO. 86/173 |
| sol | NM_000070 | Muscular dystrophy, limb-girdle, type 2A (1) | SEQ ID NO. 87/174 |
| Stat92E | NM_012448 | Leukemia, acute promyetoyctic, | SEQ ID NO. 65/152 |
| | | STAT5B/RARA type (2) | |
| Stat92E | NM_012448 | Growth hormone insensitivity with | SEQ ID NO. 65/152 |
| | | immunodeficiency (2) | |
| TSG101 | NM_006292 | Breast cancer (2) | SEQ ID NO. 67/154 |

Shown are the relevant diseases of the human homologues with a BLASTP E value of 10⁻¹⁰ or less as referenced in Homophila (Chien et al., 2002) and OMIM.
(1) data sets were downloaded from Homophila Version 2.1 (update 13 Apr 2005). Chien, S., Reiter, L. T., Bier, E. and Griboskov M., Nucleic Acids Research 30: 149-151 (2002)
(2) data sets from: Online Mendelian Inheritance in Man, OMIM (TM). McKusick-Nathans Institute for Genetic Medicine, Johns Hopkins University (Baltimore, MD) and National Center for Biotechnology Information, National Library of Medicine (Bethesda, MD), 2000. World Wide Web URL: http://www.ncbi.nlm.nih.gov/omim/
(3) information from: Kalla, C., Nentwich, H., Schlotter, M., Mertens, D., Wildenberger, K., Dohner, H., Stilgenbauer, S. and Lichter, P., Genes Chromosomes Cancer 42 (2): 128-143 (2005)

SUPPLEMENTARY TABLE 7
Expected and Observed Phenotype Frequency

| | | | Expected | | Observed | |
|---|---|---|---|---|---|---|
| | | | Phenotypes | | Phenotypes | |
| Chromosome | No Genes* | % | Pos | Neg | Pos | Neg |
| X | 2292 | 17% | 11 | 4 | 16 | 0 |
| 2L | 2444 | 18% | 11 | 4 | 10 | 5 |
| 2R | 2687 | 20% | 12 | 5 | 5 | 7 |
| 3L | 2612 | 19% | 12 | 4 | 15 | 4 |
| 3R | 3392 | 25% | 16 | 6 | 15 | 8 |
| 4 | 82 | 1% | 1 | 0 | 0 | 0 |
| Unmapped | | | | | 5 | 0 |

*Location according to Release 3.1 of the Berkeley Drosophila Genome Project

TABLE 7

| | Gene Name: | Gene Name: Homo | Accession | | SEQ ID |
|---|---|---|---|---|---|
| Ranking | D. melanogaster | sapiens | Number | Associated Disease | NOs: |
| 1 | HDC01676 | CHRNA7 | NM_000746 | Schizophrenia, neurophysiologic defect | 46/133 |
| 2 | CG4349 | FTH1 | NM 002032 | Iron overload, autosomal dominant | 29/116 |
| 3 | TSG101 | TSG101 | NM_006292 | Breast cancer | 67/154 |
| 4 | bon | TRIM33 | NM_015906 | Thyroid carcinoma, papillary | 4/_9 |
| 5 | mask | MLL3 | NM_021230 | Myeloid leukemia | 83/170 |
| 6 | enok | MYST3 | NM_006766 | Acute myeloid leukemia | 45/132 |
| 7 | Caf1 | RBBP4 | NM_005610 | Refsum disease | 68/155 |
| 8 | Rab5 | RAB5A | NM_004162 | | 86/173 |
| 9 | CG31694 | IFRD2 | NM_006764 | Small cell lung cancer | 25/112 |
| 10 | sol | CAPN3 | NM_000070 | Muscular dystrophy, limb-girdle, type 2A | 87/174 |
| 11 | CG31132 | BRODL | NM_153252 | Leukemia | 72/159 |
| 12 | CG15434 | NDUFA2 | NM_002488 | Muscular dystrophy, limb-girdle, 1A | 14/101 |
| 13 | CG3819 | ENDOGL1 | NM_005107 | Carcinomas of lung, uterus, esophagus, kidney | 207 |
| 14 | CG31005 | TPRT | NM_014317 | | 22/109 |
| 15 | Pp1alpha-96A | PPP1CC | NM_002710 | | 59/146 |
| 16 | CG10077 | DDX5 | NM_004396 | | 7/_94 |
| 17 | CG17492 | LOC142678 | NM_080875 | | 71/158 |
| 18 | kn | DKFZP667B0210 | NM_024007 | | 50/137 |
| 19 | CG31132 | C21ORF107 | NM_018963 | | 72/159 |
| 20 | Pp1alpha-96A | PPP1CA | NM_002708 | | 59/146 |
| 21 | CtBP | CTBP2 | NM_001329 | | 80/167 |
| 22 | PP2A-B′ | PPP2R5D | NM_006245 | | 60/147 |
| 23 | Nup154 | NUP155 | NM_004298 | | 57/144 |
| 24 | mask | ANKHD1 | NM_017747 | | 83/170 |
| 25 | Art2 | HRMT1L4 | NM_019854 | | 1/_88 |
| 26 | CG18112 | C14ORF133 | NM_022067 | | 19/106 |
| 27 | I(1)G0084 | PHF10 | NM_018288 | | 51/138 |

TABLE 8

| | | | z- | STAT3 | STAT1 | |
|---|---|---|---|---|---|---|
| Gene name | | | score | activity | activity | |
| Drosophila | Gene Name | Accession | [Dmel- | [induction | [induction | |
| melanogaster | Homo sapiens | Number | screen] | SOCS3] | GBP1] | Associated Disease |
| HDC01676 | CHRNA7 | NM_000746 | −2.3 | 0.6 | 1.0 | Schizophrenia, neurophysiologic defect in (2) |
| CG4349 | FTH1 | NM_002032 | −4.1 | 0.4 | 1.3 | Iron overload, autosomal dominant (2) |
| TSG101 | TSG101 | NM_006292 | 3.1 | 2.0 | 1.2 | Breast cancer (2) |
| bon | TRIM33 | NM_015906 | 5.6 | 0.4 | 1.3 | Thyroid carcinoma, papillary (2) |
| mask | MLL3 | NM_021230 | −2.3 | 1.2 | 0.5 | Myeloid leukemia (6) |
| enok | MYST3 | NM_006766 | 3.0 | 0.8 | 2.3 | Acute myeloid leukemia (5) |
| Caf1 | RBBP4 | NM_005610 | 3.0 | 1.1 | 2.0 | Refsum disease (1) |
| Rab5 | RAB5A | NM_004162 | 2.1 | 2.8 | 1.9 | |
| CG31694 | IFRD2 | NM_006764 | −2.8 | 2.2 | 2.4 | Small cell lung cancer (4) |
| sol | CAPN3 | NM_000070 | −2.5 | 2.5 | 2.0 | Muscular dystrophy, limb-girdle, type 2A (1) |
| CG31132 | BRODL | NM_153252 | −2.8 | 1.1 | 0.6 | Leukemia (3) |
| CG15434 | NDUFA2 | NM_002488 | −2.5 | 3.8 | 3.7 | Muscular dystrophy, limb-girdle, 1A (2) |
| CG3819 | ENDOGL1 | NM_005107 | −2.3 | 0.9 | 1.9 | Carcinomas of lung, uterus, esophagus, kidney (7) |
| CG31005 | TPRT | NM_014317 | −2.3 | 0.5 | 0.4 | |
| Pp1alpha-96A | PPP1CC | NM_002710 | 3.0 | 0.4 | 2.1 | |
| CG10077 | DDX5 | NM_004396 | 2.8 | 0.8 | 3.9 | |
| CG17492 | LOC142678 | NM_080875 | 2.5 | 2.4 | 2.4 | |
| kn | DKFZP667B0210 | NM_024007 | −2.4 | 2.0 | 1.3 | |
| CG31132 | C21ORF107 | NM_018963 | −2.8 | 0.5 | 0.5 | |
| Pp1alpha-96A | PPP1CA | NM_002708 | 3.0 | 0.8 | 3.2 | |
| CtBP | CTBP2 | NM_001329 | −2.9 | 0.5 | 0.8 | |
| PP2A-B′ | PPP2R5D | NM_006245 | 2.6 | 1.9 | 1.6 | |
| Nup154 | NUP155 | NM_004298 | 2.9 | 1.6 | 2.0 | |
| mask | ANKHD1 | NM_017747 | −2.3 | 0.6 | 1.9 | |
| Art2 | HRMT1L4 | NM_019854 | −2.9 | 0.6 | 2.9 | |
| CG18112 | C14ORF133 | NM_022067 | 2.1 | 1.2 | 1.7 | |
| I(1)G0084 | PHF10 | NM_018288 | −2.1 | 0.5 | 0.7 | |

References:
(1) data sets were downloaded from Homophila Version 2.1 (update 13 Apr 2005). Originally published: Chien, S., Reiter, L. T., Bier, E. and Griboskov M. Homophila: human disease gene cognates in Drosophila. Nucleic Acids Research 30: 149-151 (2002)
(2) data sets from: Online Mendelian Inheritance in Man, OMIM (TM). McKusick-Nathans Institute for Genetic Medicine, Johns Hopkins University (Baltimore, MD) and National Center for Biotechnology Information, National Library of Medicine (Bethesda, MD), 2000. World Wide Web URL: http://www.ncbi.nlm.nih.gov/omim/
(3) information from: Kalla, C., Nentwich, H., Schlotter, M., Mertens, D., Wildenberger, K., Dohner, H., Stilgenbauer, S. and Lichter, P. Translocation t (X; 11)(q13; q23) in B-cell chronic lymphocytic leukemia disrupts two novel genes. Genes Chromosomes Cancer 42 (2): 128-143 (2005)
(4) information from: Latif F., Duh, F. M., Bader, S., Sekido, Y., Li, H., Geil, L., Zbar, B. Minna, J. D. and Lerman, M. I. The human homolog of the rodent immediate early response genes, PC4 and TIS7, resides in the lung cancer tumor suppressor gene region on chromosome 3p21. Hum Genet 99 (3): 334-341 (1997)
(5) information from: Borrow, J., Stanton, V. P., Jr., Andresen, J. M., Becher, R., Behm, F. G., Chaganti, R. S. K., Civin, C. I., Disteche, C., Dube, I., Frischauf, A. M., Horsman, D., Mitelman, F., Volinia, S., Watmore, A. E., Housman, D. E. The translocation t(8; 16)(p11; p13) of acute myeloid leukaemia fuses a putative acetyltransferase to the CREB-binding protein. Nature Genet. 14: 33-41 (1996)
(6) information from: Ruault, M., Brun, M. E., Ventura, M., Roizes, G., De Sario, A. MLL3, a new human member of the TRX/MLL gene family, maps to 7q36, a chromosome region frequently deleted in myeloid leukaemia. Gene 284: 73-81 (2002)
(7) information from: Daigo, Y., Isomura, M., Nishiwaki, T., Tamari, M., Ishikawa, S., Kai, M., Murata, Y., Takeuchi, K., Yamane, Y., Hayashi, R., Minami, M., Fujino, M. A., Hojo, Y., Uchiyama, I., Takagi, T., Nakamura, Y. Characterization of a 1200-kb genomic segment of chromosome 3p22-p21.3. DNA Res. 6: 37-44 (1999)

1. A method for identifying a compound capable of modulating the activity of the JAK/STAT pathway, comprising
a contacting a compound with at least one target molecule selected from
i nucleic acid molecules, comprising
ii a nucleotide sequence as shown in SEQ ID NOs. 88 to 265;
iii a nucleotide sequence which is complementary to a nucleotide sequence of (i.1);
iv a nucleotide sequence which has an identity of at least 65% to a nucleotide sequence of (i.1) or (i.2); and/or
v a nucleotide sequence which hybridizes under stringent conditions to a nucleotide sequence of (i.1), (i.2) or (i.3); and
ii polypeptide molecules
iii encoded by the nucleic acid molecules of (i) and/or
iv having the sequences as shown in SEQ ID NOs. 1-87, and
b determining the degree of modulation of the at least one target molecule by the compound.

2. The method according to claim 1, wherein the compound is selected from compounds capable of directly and/or indirectly inhibiting or activating the transcription or translation of a nucleic acid molecule of (i).

3. The method according to claim 2, wherein the compounds capable of directly and/or indirectly inhibiting or activating the transcription or translation of a nucleic acid molecule of (i) comprise polypeptides such as proteins, enzymes, antibodies, polypeptide inhibitors, polypeptide activators, agonist, antagonists, mimetics, low molecular weight substances, antisense molecules, RNAi molecules and ribozymes.

4. The method according to claim 1, wherein the compound is selected from compounds capable of directly and/or indirectly inhibiting or activating a polypeptide molecule of (ii).

5. The method according to claim 4, wherein the compounds capable of directly and/or indirectly inhibiting or activating a polypeptide molecule of (ii) comprise polypeptides such as proteins, enzymes, antibodies, polypeptide inhibitors, polypeptide activators, agonist, antagonists, mimetics, oligopeptides, low molecular weight substances and cofactors.

6. The method according to claim 1, wherein the compound is an antibody or fragment thereof and wherein the antibody or fragment thereof is directed against a polypeptide molecule of (ii).

7. The method according to claim 1, wherein the compound is an antisense molecule and wherein the antisense molecule is directed against a nucleic acid molecule of (i).

8. The method according to claim 1, wherein the compound is an RNAi molecule.

9. The method according to claim 1, wherein the degree of modulation of the at least one target molecule by the compound is determined by measuring the amount and/or expression rate of the nucleic acid molecule of (i).

10. The method according to claim 1, wherein the degree of modulation of the at least one target molecule by the compound is determined by measuring the amount and/or activity of the polypeptide molecule of (ii).

11. The method according to claim 1, wherein the method is a molecular based assay.

12. The method according to claim 1, wherein the method is a cellular assay.

13. Use of at least one molecule selected from
i nucleic acid molecules, comprising
ii a nucleotide sequence as shown in SEQ ID NOs. 88 to 265;
iii a nucleotide sequence which is complementary to a nucleotide sequence of (i.1);
iv a nucleotide sequence which has an identity of at least 65% to a nucleotide sequence of (i.1) or (i.2); and/or
v a nucleotide sequence which hybridizes under stringent conditions to a nucleotide sequence of (i.1), (i.2) or (i.3); and
ii polypeptide molecules
iii encoded by the nucleic acid molecules of (i) and/or
iv having the sequences as shown in SEQ ID NOs. 1-87,
as a target for the modulation of the activity of the JAK/STAT pathway.

14. A method for modulating the activity of the JAK/STAT pathway comprising contacting a cell with at least one molecule selected from
i nucleic acid molecules, comprising
ii a nucleotide sequence as shown in SEQ ID NOs. 88 to 265;
iii a nucleotide sequence which is complementary to a nucleotide sequence of (i.1);
iv a nucleotide sequence which has an identity of at least 65% to a nucleotide sequence of (i.1) or (i.2); and/or
v a nucleotide sequence which hybridizes under stringent conditions to a nucleotide sequence of (i.1), (i.2) or (i.3);
vi polypeptide molecules
vii encoded by the nucleic acid molecules of (i) and/or
viii having the sequences as shown in SEQ ID NOs. 1-87, and
ii effector molecules of (i) and/or (ii).

15. The method according to claim 14, wherein the effector molecules of (i) and/or (ii) are selected from antibodies or fragments thereof which are directed against a polypeptide molecule of (ii), antisense molecules which are directed against a nucleic acid molecule of (i) and RNAi molecules.

16. A pharmaceutical composition comprising as an active agent at least one molecule selected from
i nucleic acid molecules, comprising
ii a nucleotide sequence as shown in SEQ ID NOs. 88 to 265;
iii a nucleotide sequence which is complementary to a nucleotide sequence of (i.1);
iv a nucleotide sequence which has an identity of at least 65% to a nucleotide sequence of (i.1) or (i.2); and/or
v a nucleotide sequence which hybridizes under stringent conditions to a nucleotide sequence of (i.1), (i.2) or (i.3);
vi polypeptide molecules
vii encoded by the nucleic acid molecules of (i) and/or
viii having the sequences as shown in SEQ ID NOs. 1-87, and
ii effector molecules of (i) and/or (ii).

17. The pharmaceutical composition according to claim 16, wherein the effector molecules of (i) and/or (ii) are selected from antibodies or fragments thereof which are directed against a polypeptide molecule of (ii), antisense molecules which are directed against a nucleic acid molecule of (i) and RNAi molecules.

18. The pharmaceutical composition according to claim 16, optionally containing pharmaceutically acceptable carriers, diluents and/or adjuvants.

19. The pharmaceutical composition according to claim 16 for the diagnosis, prevention or treatment of a JAK/STAT pathway associated disorder.

20. The pharmaceutical composition according to claim 16, wherein the JAK/STAT pathway associated disorder is selected from the group consisting of papillary thyroid carcinoma, Refsum disease, blood-brain barrier glucose transport defect, X-linked nonsyndromic mental retardation, long QT syndrome 4, subcortical laminar heterotopia, leukemia, steroid-resistant nephrotic syndrome, invasive pituitary tumor, sporadic Sotos syndrome, autosomal dominant iron overload, hereditary pancreatitis, stomatocytosis I, atypical Rett syndrome, phosphoglycerate dehydrogenase deficiency, Wolman disease, neurophysiologic defect in schizophrenia, autosomal recessive SCID (T-negative/B-positive type), atelostogenesis (type I), Larson syndrome, spondylocarpotarsal synostosis syndrome, frontometaphyseal dysplasia, diabetes mellitus (type II), susceptibility to insulin resistance, Griscelli Syndrome, limb-girdle muscular dystrophy (type 2A), growth hormone insensitivity with immunodeficiency and breast cancer.

21. A method for the diagnosis, prevention or treatment of a JAK/STAT pathway associated disorder comprising administering
nucleic acid molecules, comprising
ii a nucleotide sequence as shown in SEQ ID NOs. 88 to 265;
iii a nucleotide sequence which is complementary to a nucleotide sequence of (i.1);
iv a nucleotide sequence which has an identity of at least 65% to a nucleotide sequence of (i.1) or (i.2); and/or
v a nucleotide sequence which hybridizes under stringent conditions to a nucleotide sequence of (i.1), (i.2) or (i.3);
vi polypeptide molecules
vii encoded by the nucleic acid molecules of (i) and/or
viii having the sequences as shown in SEQ ID NOs. 1-87, or
ii effector molecules of (i) and/or (ii).

22. A method according to claim 21, wherein the effector molecules of (i) and/or (ii) are selected from antibodies or fragments thereof which are directed against a polypeptide molecule of (ii), antisense molecules which are directed against a nucleic acid molecule of (i) and RNAi molecules.

23. Use according to claim 21, wherein the JAK/STAT pathway associated disorder is selected from the group consisting of papillary thyroid carcinoma, Refsum disease, blood-brain barrier glucose transport defect, X-linked nonsyndromic mental retardation, long QT syndrome 4, subcortical laminar heterotopia, leukemia, steroid-resistant nephrotic syndrome, invasive pituitary tumor, sporadic Sotos syndrome, autosomal dominant iron overload, hereditary pancreatitis, stomatocytosis I, atypical Rett syndrome, phosphoglycerate dehydrogenase deficiency, Wolman disease, neurophysiologic defect in schizophrenia, autosomal recessive SCID (T-negative/B-positive type), atelostogenesis (type I), Larson syndrome, spondylocarpotarsal synostosis syndrome, frontometaphyseal dysplasia, diabetes mellitus (type II), susceptibility to insulin resistance, Griscelli Syndrome, limb-girdle muscular dystrophy (type 2A), growth hormone insensitivity with immunodeficiency and breast cancer.
